(19) United States
(12) Patent Application Publication
(10) Pub. No.: US 2024/0339226 A1
(43) Pub. Date: Oct. 10, 2024

(54) INTEGRATED HEALTH AND WELLNESS PLATFORM FOR HEALTH CARE, WELLNESS, CONDITIONING, FITNESS, AND HIGH PERFORMANCE MANAGEMENT

(71) Applicant: VITAL ECARE, San Carlos, CA (US)

(72) Inventors: Jeffrey Karl LUCAS, San Carlos, CA (US); Dixie Branner BAKER, Redondo Beach, CA (US); David Henry SKLAR, Walnut Creek, CA (US)

(21) Appl. No.: 18/292,887

(22) PCT Filed: Jul. 28, 2022
(86) PCT No.: PCT/US2022/038692

§ 371 Date: Jan. 26, 2024

Related U.S. Application Data
(60) Provisional application No. 63/226,606, filed on Jul. 28, 2021.

(51) Int. Cl. G16H 80/00; G16H 10/60; H04L 9/40

(52) U.S. Cl. CPC G16H 80/00; G16H 10/60; H04L 63/105

(57) ABSTRACT

The present application describes an electronic, self-learning platform that integrates and orchestrates services and analytics to enable collaborative, integrated planning for health, wellness, conditioning, fitness, and physical, mental, and other high-performance endeavors, progress monitoring and measurement, behavior incenting, coaching, supporting, and reporting.

### CROSS-REFERENCE TO RELATED APPLICATIONS

This application claims priority to U.S. Provisional Application No. 63/226,606, filed Jul. 28, 2021, entitled “Integrated Health and Wellness Platform for Health Care, Wellness, Conditioning, Fitness and High Performance Management,” which is incorporated by reference herein in its entirety.

### TECHNICAL FIELD

The disclosed implementations relate generally to health care management and more specifically to an integrated health and wellness platform for health care, wellness, conditioning, fitness, and high-performance management.

### BACKGROUND

Studies have established that health care management is most effective when delivered by multidisciplinary teams collaborating in both primary and public health, using a person's health and medical information, with access to health status and plans of care. Health care management goes beyond just the treatment of illness, as it also includes the management of wellness (i.e., avoidance of illness), as well as related areas, such as conditioning, fitness, athletic performance, and performance in a profession that demands peak physical and mental capability and execution.

### SUMMARY

Accordingly, there is a need for an integrated health and wellness platform for health care, wellness, conditioning, fitness, and high-performance management.

This disclosure describes techniques for implementing a highly secured and comprehensive digital platform of integrated services, deployed, and used together within a person's extended context to facilitate collaborative informed, management and improvement of health, wellness, conditioning, fitness, and/or athletic or other performance in a profession that demands peak physical and mental capability and execution. The composite, internet-based technology platform integrates 1) a repository of person-specific health and wellness related data, sometimes referred to as the Secure Personal Health Electronic Resource Exchange (SPHERE℠), aggregated from multiple sources, including healthcare encounters, fitness training, wellness training, athletic coaching, strength and conditioning training, and other health-and-wellness-related encounters within and outside the healthcare system (including financial, cost and claims data), along with assorted person-specific content generated and/or uploaded through the use of platform services; 2) a comprehensive, goal-directed, health-and-wellness management system that addresses the full set of interrelated domains critical to health and wellness-diet/nutrition, rest/sleep, exercise/activity, attitude/lifestyle/mental health, medical care, and specialist services (sometimes collectively referred to as DREAMS™, 3) a multi-media Collaboration Channel to support a team-approach to defining specific goals toward improvement of health and wellness within specified timelines; and 4) an artificial intelligence (AI) based Knowledge Engine that fosters a learning environment within a single platform implementation, for individual improvement, and across platform implementations for population-based knowledge discovery and platform improvement. Because the platform has access to, and manipulates highly sensitive, personal information relating to health, disease, genomics, wellness, fitness, athletic performance, finances, cost, claims data, etc., its resources and services are protected using strong security and privacy technology, including role-based, individual control of accesses and privileges. The primary means for privately capturing, sharing, and communicating information within the platform is based on a multi-media Collaboration Channel that provides processes and services for sharing information, experience, and content among users, including secured email, messaging, alerts, notifications, reminders, secured upload and download and real-time audiovisual collaboration. The Collaboration Channel enables uploading of any digital content from any internet-connected device, with content-recognition and transformation tools available to analyze, transform, persist, and manage the content in a standardized syntactical and semantic format for facilitated retrieval and use. The Collaboration Channel enables sharing and exchange of information, impressions, multimedia content, and feedback, aimed toward integrating and managing a person-specific DREAMS™ Plan among the individual who is the primary focus of the DREAMS™ Plan (person), the family and friends who provide ongoing support to the person in meeting the goals set forth in the DREAMS™ Plan (Personal team), the team of health and wellness professionals who provide principal support to the person (Principal Service team), the team of specialists supporting the person (Specialist team), and individuals who fulfill individual needs identified in the DREAMS™ Plan (Assigned Resources). The platform architecture is based on metadata constructs to simulate and support a project-management approach to health and wellness management, and overall individual care and improvement. Combined, the example architecture, the interrelated suite of platform services, the method of capturing and integrating a person's collective health record along with other health and wellness related information, and strong security and privacy protection, enable an efficient and effective dynamic health and wellness management environment capable of connecting a person and their collective support team with the resources needed, both internal to the platform and externally accessed by the platform, for continuous management of the most challenging health conditions, or of personal objectives toward supporting and improving health, wellness, conditioning, fitness, or performance in a profession that demands peak physical and mental capability and execution.

The techniques described herein can also be used to provide to persons with the most challenging health conditions, including those with chronic health conditions and COVID-19 “long haulers,” and to these persons' care providers and collective support team, a platform that will assist them in managing the person's health by extending care outside the clinic and into the person's daily life.

The techniques described herein can also be used to provide to persons desiring to enhance their wellness, conditioning, fitness, or athletic or other performance, and to these persons' personal, professional, specialist and other assigned team members, a platform that will assist them in managing the person's quest toward goals set forth in their DREAMS™ Plan.

The techniques described herein can also be used to provide to persons in demanding positions requiring strength, peak conditioning, and high performance on stringent daily, weekly, and monthly physical and mental tasks (e.g., professional athlete, firefighter, police) a platform through which the person is supported by a Principal Service team, Specialist team, Personal team, and Assigned Resources collectively directing, coaching and supporting intricate and interrelated activities aimed to achieve both individual goals and, in some cases, team goals.

The techniques described herein can also be used to collect and integrate relevant health, wellness, and physical and mental conditioning information into a single SPHERE℠, to include records pulled from external sources, as well as information collected within the platform itself, such as social determinants of health, home-health outcomes, health Internet of Things (IoT) device data, and responses to surveys generated within the platform.

The techniques described herein can also be used to provide technology capable of receiving virtually “any” digital content the person or collective support team might choose to upload, inspect the content for human and platform safety, and transform that content into structured, metadata-tagged data, rendering it findable, retrievable, interpretable, and actionable within the platform context.

The techniques described herein can also be used to provide a dynamic, collaboration, communications channel that offers private and secured, multi-media services to enable the person, the Principal Service team, Specialist team, Personal team, and Assigned Resources to communicate in real-time (e.g., synchronous, audio-visual collaborations), or through asynchronous messaging (e.g., secured email, text messaging) and content exchange (e.g., content upload), and that provides triggered notifications (e.g., reminders, outcome results, feedback) to the person, Personal team, Principal Service team, Specialist team, and Assigned Resources as appropriate.

The techniques described herein can also be used to provide a dynamic, transparent mechanism for translating a person's one or more plans prescribed by primary care providers (PCPs), specialists, coaches, and other professionals into a single DREAMS™ Plan that articulates specific, measurable goals in the health domains of diet/nutrition, rest/sleep, exercise/activity, attitude/lifestyle/mental health, medical care, and specialist services, along with support needs and timeline for meeting goals.

The techniques described herein can also be used to provide metrics, methods, and technologies for motivating and measuring progress toward meeting DREAMS™ Plan goals and positive outcomes in all health, wellness, conditioning, fitness, or high-performance domains.

The techniques described herein can also be used to provide a robust Knowledge Engine that consumes identified data to help assess and provide feedback, recommendations and updates to each person's current status, improvements, outcomes and DREAMS™ Plan; and consumes and analyzes anonymized/deidentified data across individual platform deployments, and the entire population of platform users, to provide insights to the Person, the Personal team, the Principal Service team, the Specialist team, and Assigned Resources as appropriate and to enable continuous improvement of the power of the platform.

The techniques described herein can also be used to provide to the Person, the Principal Service team, Specialist team, Personal team, and Assigned Resources appropriate and tailored feedback that they can use to adjust the DREAMS™ Plan to further mold behavior toward adherence and improved outcomes, and to present this feedback within the context of a customized dashboard providing an overview of the current state and progress within each role's realm of influence; e.g., the Person's dashboard displays only the Person's performance and progress, whereas the Principal Service team's dashboard summarizes outcomes for all Persons who are under their care and who have authorized the individual to view their data.

The techniques described herein can also be used by the Person, Personal team, Principal Service team, Specialist team and Assigned Resources to understand the Person's health and wellness related financial status, claims data and costs in order to support budgeting and planning of the Person's care.

The techniques described herein can also be used to provide in the platform architectural features and functional services to protect against accidental and/or intentional security threats to the confidentiality, accuracy, and availability of the data and services.

The techniques described herein can be used to provide in the platform architectural and functional features to enable an individual Person and/or their assigned proxy to manage access to their personal data, and to assign individual roles and rights to each individual on their collective support team.

In another aspect, a system configured to perform any of the above methods is provided, according to some implementations.

The present application discloses subject-matter in correspondence with the following numbered clauses:

(A1) A method for health and wellness management, comprising: generating a health and wellness plan for a Person that includes (i) a diet plan, (ii) a rest plan, (iii) an exercise plan, (iv) an attitude/lifestyle/mental health plan, (v) a medical care plan, and (vi) specialist services, based on a repository of health and wellness data that is specific to the Person; selecting a plurality of individuals to form a support network for the Person based on the health and wellness plan; assigning roles, responsibilities and role-based access rights for the plurality of individuals in the support network, based on the health and wellness plan; and reporting and updating (i) the health and wellness plan for the Person, and (ii) the roles, responsibilities and role-based access rights for the plurality of individuals, based on tracking health of the person.

(A2) The method as recited in clause (A1), further comprising: generating the repository of health and wellness data for the person by integrating the person's health-system records with other digital content related to the person's health, wellness, conditioning, fitness, athletic performance, or professional physical, mental or other performance gathered from external sources, including care providers, athletic trainers, coaches, labs, specialists, and other individuals and organizations, along with genomic data, social determinants of health (SDOH), device data, financial, cost and/or claims data, and other person-generated data provided through uploads, responses to surveys generated, and outcomes feedback obtained during execution of the health and wellness plan

(A3) The method as recited in any of clauses (A1)-(A2), further comprising: providing an always-on, private collaboration channel that enables sharing and exchange of content, for integrating and managing the health and wellness plan among the person and the plurality of individuals in the support network.

(A4) The method as recited in any of clauses (A1)-(A3), further comprising: using an artificial intelligence (AI)-based knowledge engine to: (i) continuously receive data for the person, analyze a current state, identify data omissions and potential adverse events, and (ii) generate insights for modifying and/or fulfilling the health and wellness plan based on the current state and identified data omissions and potential adverse events; and providing feedback to the person and the plurality of individuals, based on the generated insights.

(A5) The method as recited in any of clauses (A1)-(A4), further comprising: analyzing, using an AI-based knowledge engine, anonymized data across a population including the person, to obtain population-level knowledge; and providing feedback to the person and the plurality of individuals, based on the population-level knowledge.

(A6) The method as recited in any of clauses (A1)-(A5), further comprising: measuring progress towards meeting goals of the health and wellness plan; and providing feedback to motivate the person and modify daily behavior and activities of the person, to meet the goals, based on the measured progress.

(A7) The method as recited in any of clauses (A1)-(A6), further comprising: integrating and orchestrating services and analytics to enable collaborative, integrated planning for health, wellness, fitness, and athletic-performance, change monitoring and measurement, behavior incenting, coaching, supporting, and reporting both internal to the platform and to external authorized recipients.

(A8) The method as recited in any of clauses (A1)-(A7), wherein the health and wellness plan includes one or more goals and associated timelines.

(A9) The method as recited in any of clauses (A1)-(A8), wherein assigning the roles, responsibilities and role-based access rights comprises: scheduling tasks, events and activities with calendar-based planning and management tools; managing task dependencies and task interdependencies; assigning of task, event and activity owners; and tracking of progress and completion of tasks, events and activities to support notification of appropriate users.

(A10) The method as recited in any of clauses (A1)-(A9), further comprising: applying a rules-based decision algorithm, based on the health and wellness plans, the person's health system records and real-time health and wellness information, to trigger one or more updates selected from the group consisting of: (i) generating system alerts and providing notifications regarding missing information; (ii) identifying changes to the health and wellness plans, the person's health system records, and the real-time health and wellness information; (iii) notifying appropriate users when tasks, events and activities are completed, on pace with or falling behind expected progress, or that need to be completed; (iv) alerting the person and other authorized users of changes in team composition and/or role/access capabilities; and (v) suggesting new activities or changes to activities based on the rules-based engine and updated data.

(A11) The method as recited in any of clauses (A1)-(A10), further comprising: performing receipt, recognition, transformation, metadata tagging, and persistence of any structured or unstructured digital content.

(A12) The method as recited in any of clauses (A1)-(A11), further comprising: providing an interface to assign the roles, responsibilities and role-based access rights to data and services to individuals of the plurality of individuals; and receiving, via the interface, the roles, responsibilities and role-based access rights to data and services.

(A13) The method as recited in any of clauses (A1)-(A12), further comprising: providing a summary snapshot of current state and progress for each individual of the plurality of individuals and the person, in accordance with the role-based access rights to data and services assigned to each individual.

(A14) The method as recited in clause (A13), wherein the summary snapshot is categorized based on whether an individual belongs to the person's family and friends, a healthcare team, or a specialist team.

(A15) The method as recited in any of clauses (A1)-(A14), further comprising: applying information security measures for protecting confidentiality, integrity and availability of data and services available to the person and the plurality of individuals.

(A16) The method as recited in any of clauses (A1)-(A15), The method of claim 1, further comprising: for each person of a plurality of persons: generating a respective health and wellness plan based on a respective repository of health and wellness data; selecting a respective plurality of individuals to form a respective support network for the respective person; assigning respective roles, responsibilities and role-based access rights for the respective plurality of individuals in the support network, based on the health and wellness plan; and reporting and updating (i) the respective health and wellness plan for the person, and (ii) the respective roles, responsibilities and role-based access rights for the plurality of individuals, based on tracking health of the respective person.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures.

FIG. 1 is a functional diagram of the overall architecture, principal components, and high-level interactions, built upon strong security and privacy protections, according to some implementations.

FIG. 2 is a functional diagram depicting the interdependent workflows between the individual players, the SPHERE℠, and the central DREAMS™ Engine according to some implementations.

FIG. 3 is a block diagram illustrating the workflow for capturing data, pre-processing data, transforming, and persisting structured, normative data, and analyzing, transforming, and loading non-normative data into the SPHERE℠ according to some implementations.

FIG. 4 is a block diagram depicting how the Collaboration Channel enables all players to collaboratively help manage the person's health according to some implementations.

FIG. 5 is a block diagram depicting the functional structure of the DREAMS™ Engine and its participants, functions, and interactions according to some implementations.

FIG. 6 is a block diagram depicting the Knowledge Engine and its interactions according to some implementations.

FIG. 7 shows an example visualization of a DREAMS™ plan summary, according to some implementations.

FIG. 8 shows an example visualization of a DREAMS™ planner, according to some implementations.

FIG. 9 shows an example user interface for the configuration of role-based access controls by users, according to some implementations.

FIG. 10 shows example graphs for tracking specific goals and activities, according to some implementations.

FIG. 11 shows an example user interface for updating a DREAMS™ plan, according to some implementations.

FIG. 12 shows an example user interface for creating surveys, according to some implementations.

FIG. 13 shows an example user interface of a calendar-based planning tool for needs activities, according to some implementations.

FIG. 14 shows another example user interface for the configuration of role-based access controls by users, according to some implementations.

FIGS. 15-18 show example visualizations of summaries for different roles, according to some implementations.

### DESCRIPTION OF IMPLEMENTATIONS

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

It will also be understood that, although the terms first, second, etc. are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The techniques described herein are architected, designed, and built to create an environment that extends beyond the environment in which a Person receives care, training, or coaching (e.g., hospital or clinic, fitness studio, athletic training facility) and into the Person's home, office, favorite walk, or any other location, providing continuous virtual support and care, and continuously measuring progress toward reaching goals established in the person's DREAMS™ Plan, with continuous and contiguous connection to resources and the various team members and Assigned Resources involved in, and supportive of, the goals established in that DREAMS™ Plan. Described herein is a cloud-based, digital-health platform that provides each person and their collective support team with always-on, secured, private access to a powerful suite of collaboration, behavior management, roles management, health management, and content management tools, along with a comprehensive, longitudinal, aggregated Secure Personal Health Electronic Resource Exchange (SPHERE℠), seamlessly integrated into one service-centric architecture, supported by an artificial intelligence (AI) driven learning component. The techniques described herein are designed and architected to provide Persons who suffer from acute and/or chronic health conditions, and their authorized collective support team with unparalleled, integrated management capabilities centered around whole-person care anytime, anywhere, with all vital resources needed for effective care-management and recovery, conveniently available from any Internet-connected mobile device or web browser. For Persons without acute or chronic health conditions who wish to manage their overall health, wellness, conditioning, fitness, athletic performance, or performance in a profession that demands peak physical and mental capability and execution, the techniques described herein provide convenient, always-on access to an integrated set of resources and services to support their personal objectives. Persons are able to invite and authorize online role-based access to their Personal team of family and friends, and to their team of professionals and specialists, including primary care providers, coaches, athletic trainers, nutritionists, and other community resources, enabling them, at their convenience, to collaborate, support and engage in the Person's daily, weekly, and monthly efforts toward achieving the goals set forth in the DREAMS™ Plan. The techniques described herein empower each Person (or their proxy) to determine and assign the appropriate roles and rights to each individual in their collective support team involved in their care and want in their care circles and care communities.

As a Person builds their SPHERE℠ of personal information, the platform enables interoperable exchange of any and all health and wellness related information, combined with the ability to capture and store structured and unstructured digital content into the pre-designated, metadata-defined areas of the SPHERE℠ content-management architecture. The attributes of the SPHERE℠ architecture are a proprietary scheme for capturing any digital structured or unstructured content (e.g., imported health records, uploaded files and images, videos, voice messages), tagging the content with metadata for efficient indexing and retrieval, and discretely assigning access and control capabilities to other users. Each medical team member in a Person's continuum of care, such as PCP and specialists, is able to contribute to the Person's complete SPHERE℠ and dynamic DREAMS™ Plan. Each unique provider or contributor to the SPHERE℠ has the record of data they contribute discretely imported for the Person to view or assign to other authorized users to access on an “as needed” basis in order to effectively engage and collaborate in the person's care, using tools integrated into the platform.

The planning and management process implemented by the services provided by the platform is driven by a project management (PM) structure and fed by team-based collaboration tools to give authorized users access to the applicable DREAMS™ Plan to assess the composite needs. The DREAMS™ Plan generally states a Person's needs in six domains generally associated with health and wellness: 1) Diet/nutrition, 2) Rest/sleep, 3) Exercise/activity, 4) Attitude/lifestyle/mental health, 5) Medical care, and 6) Specialist services. Each person is unique, and whole-person and patient-centered care requires assessment of the person to exact the right combination of goals, objectives and plans in each of these six domains. Additionally, any one of the six domains ideally needs to be considered in concert with the other domains. For example, diet and nutrition needs cannot effectively be determined unless the Person and their Personal team understand any medication effects, level of exercise, any rest/sleep issues, lifestyle or food insecurities, and recommendations from any of the other specialists. These domains all come into consideration when collaborating and determining the most advantageous DREAMS™ Plan for each unique Person. Additionally, access to all of the Person's health background and profile enables the individuals in the Person's collective support team to support the Person in making the right decisions based on principals of “whole person” or “person centered” care concepts in each domain, using information contained in the Person's SPHERE℠.

The DREAMS™ Engine includes a Virtual Health Assistant (driven by the Knowledge Engine) that can be used to scan the Person's SPHERE℠ to check and verify that key data are present in the Person's profile and can alert or send notice to the Person if data are missing and need to be provided in order to best position the collective support team in making the optimal recommendations. Further, the Virtual Health Assistant continuously scans the Person's SPHERE℠, based on prescribed rules, to highlight and promote alerts, reminders, recommendations, and other messages to the various team members to quickly be made aware of key factors to consider. Then using the project management tools and calendar, they can effectively create or update a detailed DREAMS™ Plan designed specifically for the Person, based on the comprehensive assessment, and can personalize a calendar for completing DREAMS™ Plan tasks and activities. Each Personal team member can be assigned to support and monitor the DREAMS™ Plan or parts of it to help the Person adhere to the overall DREAMS™ Plan by providing support and engagement online. The project-management, calendar-based, care-planning tools help to schedule a Person's detailed DREAMS™ Plan on a daily, weekly, and monthly basis. This enables informed decisions for personalized daily, weekly, and monthly activity, with continuous progress monitoring and feedback. This may include scheduling exercise routines, doctor appointments, tracking diet and nutrition and other activities in the DREAMS™ Plan.

The platform also allows Persons and Personal teams to coordinate support needs, such as transportation to appointments, delivery of meals, monitoring and tracking of medication adherence and more. As the Person prepares for clinic visits, the Person can authorize the platform to electronically send personal health information requested by the Principal Service team or Specialist team. At any time, any authorized role can access the platform and view an information dashboard that summarizes the progress and status of the Person for whom they are authorized. After each encounter with the healthcare system, health record updates from the visit are automatically or manually uploaded to the Person's SPHERE℠, and notification is sent to the Personal team members for review. Extensive alerts, notifications, messaging, and content management combine to enable the kind of comprehensive, collaborative, and timely care essential for persons living with acute and/or chronic conditions, as well as healthy individuals desiring continuous monitoring and improvement of their health, wellness, conditioning, fitness, athletic performance, or profession that demands peak physical and mental capability and execution.

The techniques described herein apply to all of these and other areas that ultimately depend upon health and wellness, and henceforth will assume to be included in the umbrella term “health and wellness.”

The techniques described herein offer an integrated platform of services, content-management efficiencies, collaboration facilitation, health and wellness management services, and knowledge-management capabilities, deployed collectively to prescribe a DREAMS™ Plan, monitor and measure outcomes toward fulfillment of that Plan, and incent positive health and behavior change. Furthermore, engaging individuals and families in a Person's day-to-day health and wellness activities, and in developing and managing a goal-directed plan, helps improve health and wellness, reduces costs, helps improve individual outcomes, and helps improve overall quality of life. No platform is currently available to fully support all such engagements in a holistic way. Furthermore, as the interest and engagement of telemedicine, virtual health and wellness coaching, personal genomics, and consumer health monitoring technology continue to increase, we are experiencing a shift from dependence upon the healthcare's “in clinic” care, to supporting individuals' health and wellness wherever they may be—in their homes, at work, and in their communities. The healthcare system is recognizing the value of these “remote” extensions to services, as demonstrated by the Centers for Medicare & Medicaid Services (CMS) requirement that healthcare providers offer patient portals, and new incentives for telemedicine encounters and health coaching. At the same time, the 21^{st} Century Cures Act Final Rule of 2020 requires that healthcare organizations expose application programming interfaces (API) that enable patients to use personal apps to query for and retrieve their own healthcare data, and imposes significant penalties on healthcare organizations that attempt to block access to patients or other providers. Thus the US regulatory environment has paved the way toward widespread use of technology like that offered by the techniques described herein.

Including Personal teams in more real-time assessment of a Person's health and wellness needs, and helping the Person adhere to a goal-directed DREAMS™ Plan, are vital to improving outcomes and overall quality of life. Perhaps the most urgent need for the US healthcare system's use of the techniques described herein may be in the everyday monitoring and care of the ever-increasing number of individuals dealing with chronic conditions—a challenge exacerbated by the COVID-19 “long haulers.” The primary stakeholders in chronic-care management besides the patient, such as healthcare providers, insurers, and healthcare delivery organizations, are incented to invest in comprehensive virtual care and tools to help improve patient and family engagement, and efficient care collaboration across the continuum of care. However, no comprehensive solution exists for providing virtual care in a highly accessible, scalable, private, and secured environment that includes tools to support collaboration, planning, and communication with stakeholders. Such a solution is essential in order to accomplish ubiquitous, virtual care, that includes patient and Personal team engagement. The design and architecture described herein has uniquely integrated multiple services to provide a scalable platform for delivering a seamless and comprehensive solution for the future as envisioned to manage across the spectrum of wellness, illness, recovery, and peak performance.

Even though hospitals and clinics are more proficient and effective today, it is the moment the patient leaves the healthcare system environment that the real challenge begins. It is important in our society today to be able to support members of society challenged by chronic health conditions. The ability to monitor and measure behavior and adherence to plans of care issued at the point of care remains an unmet need that should be part of all future healthcare solutions for achieving healthier societies. No tools exist today to effectively engage patients, Personal teams, Principal Service teams, Specialist teams, and Assigned Resources. Nor is there a single platform that collects and manages an individual's complete health record, including medical records, payment information, and patient-provided information, along with support services enabling users to access, view, and manage information, and engage in collaboration, communication, and coordination of health-management events. The techniques presented herein enable fluid and seamless continuity of whole-person-centered care across the care continuum and within the individual's care community, supporting the achievement of health, wellness and fitness goals for people and populations. The platform's role-based controls help assure that each user is authorized to access only the data and services needed to perform their assigned role, both individually and as a member of the collective support team responsible for the management and improvement of the Person's health, as defined by the DREAMS™ Plan. With the innovations described in this patent application, all of these separate and discrete functions can be designed and integrated into a single platform, enabling people to have real-time ability to plan and attend to daily, weekly, and monthly care and wellness for a better, healthier life for all persons using the platform.

Concepts presented in this disclosure may also be applied to the high-performance and competitive world of collegiate and professional sports. Sports provide a classic example of the need for communications, collaboration, assessment of readiness and skill of players to help accomplish improvement for each player to achieve individual and/or team success. Just as the techniques described herein may be applied to caring for individuals with chronic conditions, the same collaborative, development planning, and performance monitoring and measurement can be used to enable teams of people to access relevant data and content, collaborate, prescribe, monitor, and measure progress toward individual athletic skill development and team performance. Given the need to develop players, coaches are challenged to acquire a comprehensive view of each athlete and a vision and plan for the development of each individual player and the team as a collective. This includes evaluating skill, strength, endurance, agility, mental resilience, and other attributes that collectively make for a skilled and successful athlete who can perform at the highest level, keeping the team competitive. Once each player is charted, a DREAMS™ Plan may be put in place to improve readiness and skills, toward achieving peak performance. The broad scope of the DREAMS™ Plan includes the same key domains described above: Diet/nutrition, Rest/sleep, Exercise/activity, Attitude/lifestyle/mental health, Medical care, and Specialist services. Helping the player work toward attainment of goals established in the DREAMS™ Plan requires the coaches, trainers, individual players, and their collective support team to collaborate toward both individual and team improvement. Working toward the DREAMS™ Plan demands that all coaches see the individual holistically and collaborate to ensure the right balance is coordinated across the professional coaching staff—from head coach to nutrition coach to academic coach to position coach.

The healthcare, fitness, wellness, and sports environments collectively inspired the vision for the health-and-wellness techniques described herein. The platform presented here provides the data services, content-management services, project-management services, knowledge management, and behavior development support services needed for person-centered planning, goal setting, and physical and mental outcomes improvement, guided by a DREAMS™ Plan. Each DREAMS™ Plan is tailored to the individual's needs in the key areas of personal improvement: Diet/nutrition, Rest/sleep, Exercise/activity, Attitude/lifestyle/mental health, Medical care, and Specialist services. Each DREAMS™ Plan is developed through a collaborative effort involving the Principal Service team, Specialist team, Personal team, the Person, and Assigned Resources, who are collectively responsible for supporting the Person's development, whether the development goals focus on health, wellness, conditioning, fitness, athletic performance, or performance in a profession that demands peak physical and mental capability and execution. The heretofore disparate means of sharing data, messages, documents, calendar, and performance metrics are presented as one seamless, supporting platform for collaboratively developing the DREAMS™ Plan, and monitoring, measuring, and incenting progress toward meeting the DREAMS™ Plan goals.

Some implementations help extend the hospital or clinic into the Person's home, office, favorite walk, or wherever the Person may be. The platform is used by the Person, along with their authorized Principal Service team, Specialty team, Assigned Resources and members of their Personal team in collaboratively developing a DREAMS™ Plan, with goals and timeline, monitoring and measuring progress toward meeting goals, incenting the Person toward achieving DREAMS™ Plan goals, and continuously learning new insights into the Person's own health and behavior, and into trends in the population of people using the techniques described herein.

### Example Platform Architecture

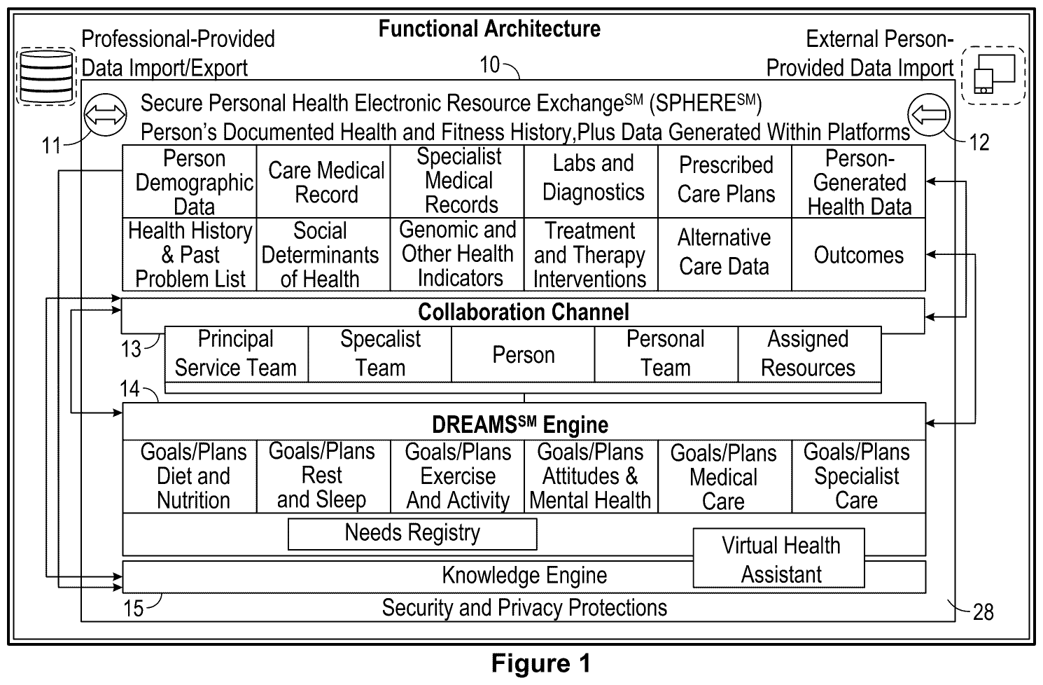

FIG. 1 illustrates an example platform architecture, according to some implementations. The architecture comprises four discrete components, integrated into a platform as interdependent and interactive:
1. A Secure Personal Health Electronic Resource Exchange (SPHERE℠) 10 is both collector and manager of person-related health information, and information provisioner for the platform services and to authorized external repositories 11. This repository includes content pulled from external sources (e.g., core medical record [i.e., elements defined in Fast Healthcare Interoperability Resources (FHIR) U.S. Core Profiles], genomic data, healthcare financial data) 11, content generated by the Person (e.g., Health IoT devices, person inputs to forms and surveys, consumer-genomics data) 12, content generated through the Collaboration Channel (e.g., DREAMS™ Plan) 13, and insights generated by the and DREAMS™ Engine 14.
2. A Collaboration Channel 13 is the dynamic, multimedia, collection of services that synergistically provide both synchronous and asynchronous communications and sharing among the Person, Principal Service team, Personal team, Specialist team and Assigned Resources with support from the DREAMS™ Engine 14 and the Knowledge Engine 15.
3. A DREAMS™ Engine 14 component includes services that support the Person and their team in collectively agreeing upon Dietary/nutrition, Rest/sleep, Exercise/activity, Attitude/lifestyle/mental health, Medications/therapies, and Specialty providers/health specialists (DREAMS™) goals that are most realistically achievable and measurable within an established timeline, along with the support the Person may need in meeting the established goals (e.g., transportation, multimedia content, personal support). In addition, this component provides services to remind the Person to perform the tasks assigned in the DREAMS™ Plan, measure progress, and provide feedback and summary analysis data on a predetermined basis defined by the user.
4. A Knowledge Engine 15 accumulates person-specific measurable actions and outcomes to provide summary data to the Person, Principal Service team, Specialist team, Personal team, and Assigned Resources via the Collaboration Channel 13. In addition, the knowledge component accumulates anonymized data 25 for use in population-wide studies whose insights are made available for use in improving platform services and performance, or toward the advancement of knowledge outside the platform environment. The Knowledge Engine can also be directed to enforce or guide use of the platform based on person-specific rules (e.g., food allergies, insurance coverage rules) or installation-specific rules.

Because the platform contains very sensitive and private health and wellness information, and offers powerful services relating to individual health and population intelligence, the platform must be implemented using strong security and privacy architectural features and functional services 28, including encryption of data at rest, encrypted links for connecting to the platform, and strong role-and-rights management 28. Any implementer should implement the platform in compliance with all applicable privacy and security laws and regulations, including, for US installations, the provisions of the Security and Privacy Rules issued pursuant to the U.S. Health Insurance Portability and Accountability Act (HIPAA), and for platform implementations used by persons outside the U.S., the security and privacy provisions of the European General Data Protection Regulation (GDPR), as well as any applicable local requirements.

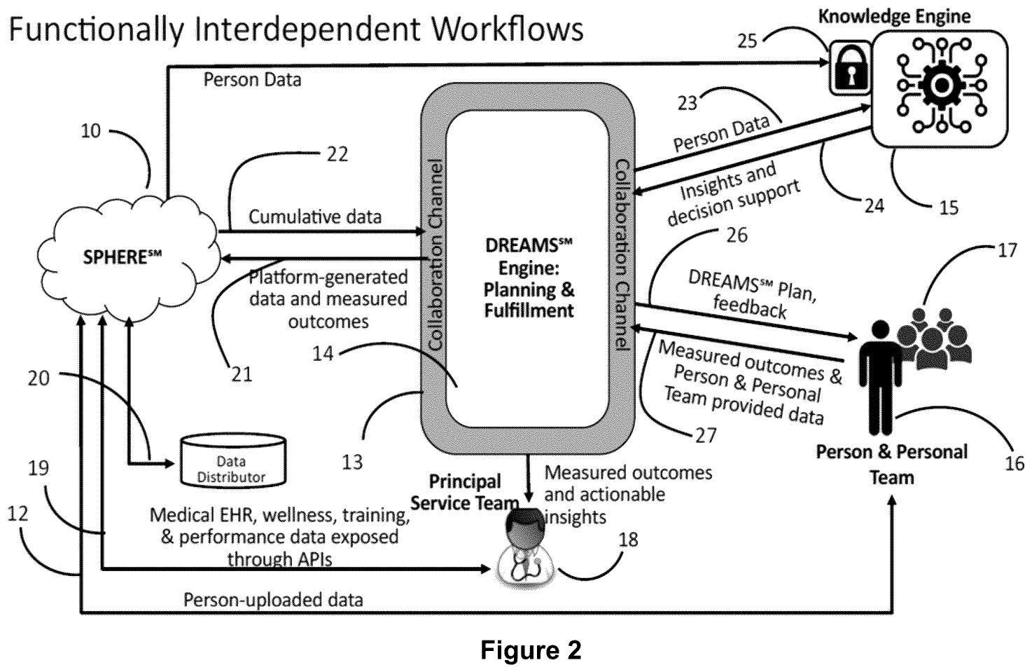

As shown in FIG. 2, these four discrete and independent components (SPHERE℠, Collaboration Channel, DREAMS™ Engine, and Knowledge Engine) are integrated within the platform as interdependent and interactive, each component relying on the others, with new knowledge discovered by the Knowledge Engine 15 being integrated into the SPHERE℠ 10 to support the Collaboration Channel 13 and DREAMS™ 14 planning and fulfillment. At the core of the example platform are the Collaboration Channel 13 and DREAMS™ Engine 14 planning and fulfillment. Interdependent with the Collaboration Channel and DREAMS™ Engine are the SPHERE℠ 10, Knowledge Engine 15, Person 16, Personal team 17, and Principal Service team, Specialist team, and Assigned Resources 18. The SPHERE℠ accumulates electronic health record (EHR) data and other relevant person-specific data by querying application programming interfaces (APIs) exposed by Principal Service team, Specialist team, and Assigned Resources 19 and from intermediary data distributors 20, from uploads from the person 12, and platform-generated data 21 through the DREAMS™ Engine and the Collaboration Channel (e.g., social determinants of health [SDOH], Health IoT devices used by the person, outcomes, healthcare financial data), and provisions accumulated and normalized data 22 to the DREAMS™ Engine 14 and Collaboration Channel 13. The Knowledge Engine 15 receives person-specific data 23 from the DREAMS™ Engine and the SPHERE℠, and returns person-specific insights 24 to the DREAMS™ Engine and Collaboration Channel. For population-based analyses, person-specific data received from the SPHERE℠ 10 are anonymized or deidentified 25 in accordance with application policy and regulations and combined with anonymized data from other platform implementations, for use in discovering new insights that are then made available to all instances of the platform. Anonymization is defined differently under different rules and regulations. For example, the United States HIPAA Privacy Rule specifies deidentified instead of anonymized data, unlike European countries, and anonymization is less than deidentification.

The Person and Personal team 16 receive a consolidated DREAMS™ Plan 26 that defines goals, needs, and timeline from the DREAMS™ Engine and the Collaboration Channel; and they return to the DREAMS™ Engine measured outcomes as well as health data 27. In turn, the DREAMS™ Engine returns to the Person constructive feedback, or notifications for areas where compliance needs improvement 26. Through the Collaboration Channel 13, the Principal Service team, Specialist team, Personal team, and Assigned Resources 18 receive measured outcomes and actionable insights through an individualized dashboard that includes the specific measures each Person requests through settings in their platform instance designed for person and population health, wellness, conditioning, and fitness management. In turn, each Principal Service team Specialist team and Assigned Resource 18 updates the Person's medical data and the care plan maintained by the practice—and changes are ultimately uploaded to the SPHERE℠ through its external-data-import services 19, 20.

### Example Secure Personal Health Electronic Resource Exchange (SPHERE℠)

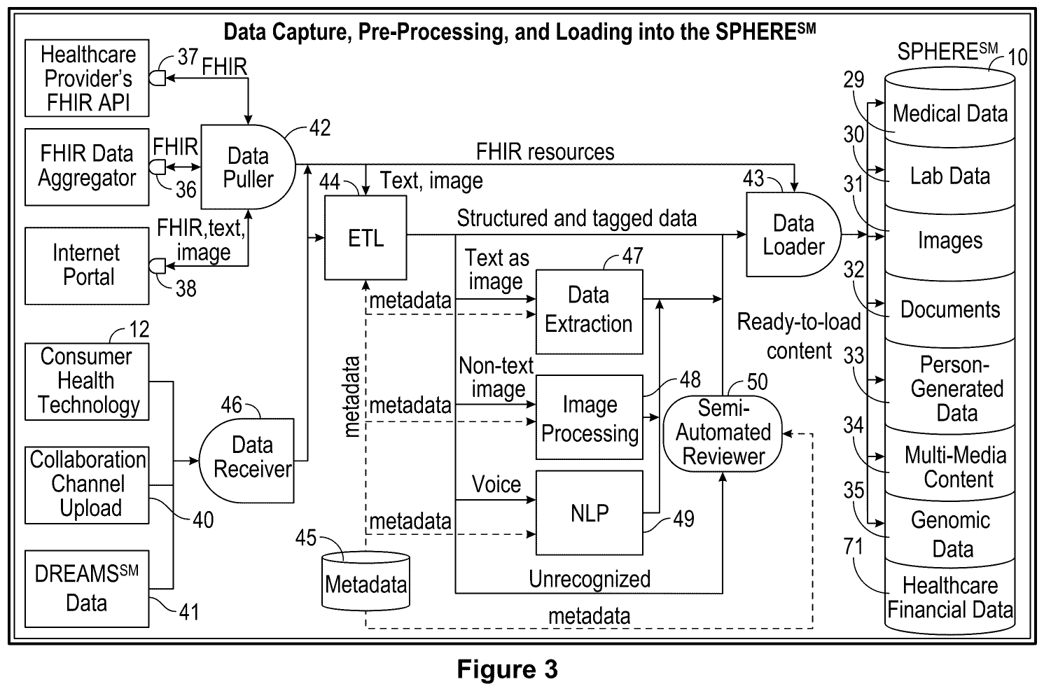

FIG. 3 depicts the data flow for populating the SPHERE℠ 10, a dynamic, multi-media repository comprising the entire collection of health, wellness, conditioning, fitness, and performance related content associated with the Person, along with services needed to collect, store, manage, and retrieve this information. The SPHERE℠ is designed for extensive capture of a person's composite and longitudinal health, wellness, conditioning, fitness, and performance related information from any source generated external or internal to the platform, along with multi-media content uploaded through the Collaboration Channel 13 for use in fulfilling that Person's DREAMS™ Plan.

As shown in FIG. 1, the SPHERE℠ 10 contains a number of categories of health, wellness, conditioning, fitness, lifestyle, and performance related information. All of the data in SPHERE℠ are indexed and persisted using the Health Level Seven International (HL7) Fast Healthcare Interoperability Resources (FHIR) standard (https://www.hl7.org/fhir/overview.html), including core medical data, person-generated data, healthcare financial data, and multi-media content, and are efficiently stored according to media type. As examples:
FHIR core medical resources imported from an EHR are persisted with other structured medical data 29;
Results of a Complete Blood Count (CBC) are persisted with other FHIR lab resources 30;
Diagnostics may include a PET positron emission tomography (PET) scan image, which are persisted as FHIR media resources along with other image results 31;
A document a PCP wants to make available for online viewing or download to persons are persisted as a FHIR document and stored with other documents 32;
Results from a survey generated by the DREAMS™ Engine are be persisted with other person-generated resources 33;
A video a PCP wants to make available for viewing or download to person is persisted as FHIR media resources and stored with other multimedia content 34; and
An exome sequence generated by 23andMe is stored with genetic data 35.
The Person's financial information and cost and claims data from healthcare related encounters with the Person is stored as defined in the FHIR financial module 71.

Today's US healthcare environment includes very little genomic data, other than specific genetic lab test results, such as those for BRCA1 and BRCA2 screening (persisted as lab data 30). However, it is anticipated that in the future, whole genome sequencing is likely to be performed as a standard procedure, particularly as the cost of sequencing continues to fall. This is already true internationally as more countries are sequencing patients routinely as part of their clinical record. Therefore, the SPHERE℠ platform includes genomic data storage 35, which may in the short term include consumer-genomics sequencing data, from sources such as 23andMe.com, Helix.com, Ancestry.com, and others. Because of the volume of genomic data, these data are likely to be imported via physical media.

As shown in FIG. 3, health, wellness, conditioning, fitness, and performance related data (structured and unstructured) will come into the platform via the following six primary channels:
1. As structured and coded medical and healthcare financial data pulled from APIs exposed by electronic health record (EHR) aggregators 36;
2. As structured and coded EHR data pulled from APIs exposed by individual healthcare providers 37;
3. As data pulled from internet portals holding the Person's personal health and wellness data 38;
4. As structured, data imported from consumer health devices 12 (e.g., Fitbit, Apple Health, Oura);
5. As content uploaded through the Collaboration Channel 40; and
6. As data generated by the DREAMS™ Engine, including response data extracted from structured surveys and measured outcomes from fulfillment of the DREAMS™ Plan 41.

The Data Puller 42 imports data from electronic health records (EHR) primarily through an EHR data aggregator 36 that accumulates, cleanses, and manages person-specific EHR data, and exposes an application programming interface (API) through which FHIR resources may be accessed. Alternatively, the Data Puller may import FHIR resources directly from a healthcare provider's source EHR, lab, or other professional's system exposing a FHIR API 37. Processing of FHIR resources is relatively straightforward, as most of the data will use standard semantics and syntax. FHIR resources include Patient Demographic Data, Health History (family and person), and Problem Lists (past and current), Core Medical data, Treatment and Therapy Interventions, Lab and Diagnostic data, as well as resources for media and complete documents. Similarly, surveys constructed using DREAMS™ Engine services will capture data as FHIR resources, using the platform's standard data and metadata models.

Some health and wellness professionals (e.g., clinics, specialists, labs, athletic trainers) may enable electronic data access through web-based portals 38, in which case the data may be FHIR resources, structured text (e.g., TXT, CSV, XML), or a text image (e.g., PDF). The Data Puller recognizes FHIR resources and transmits them directly to the Data Loader 43, which loads them into the SPHERE℠ 10. The Data Puller 42 directs non-FHIR data to the extract, transform, and load (ETL) technology 44, which first performs a safety and security check, then extracts recognized data elements, normalizes the data, and attaches metadata tags 45 to transform the data into FHIR resources, and forwards them to the Data Loader 43 (via ETL technology 44), which persists the data in the appropriate data store. All data persisted in the SPHERE℠ are metadata-tagged 45 with such attributes as data type (i.e., container), date received, and provenance.

In addition to pulling health-and-wellness service providers' records and alternative treatment data through Data Puller 42 import, information also may be uploaded through the Collaborations Channel 13, such as unstructured text, images, voice or video recordings, or other digital content. Because these data may include exercise, nutrition, treatment, or therapy interventions, it is important that the platform be able to accept, tag, and persist any data it receives through any channel (e.g., uploaded, generated in response to a DREAMS™ survey), and that the DREAMS™ Engine 14 be able to ingest the content as information usable for the person's fulfillment of their DREAMS™ Plan. All content uploaded to the platform via consumer health technology 12, Collaboration Channel upload 40, or generated by the DREAMS™ Engine 41 are directed to the Data Receiver 46, which prescreens the content for security and format. All data generated by the DREAMS™ Engine will already be structured and metadata-labeled and are sent by the Data Receiver 46 directly to the Data Loader 43. The Data Receiver 46 directs all other data to the ETL technology 44, which extracts recognized data elements, tags them with metadata 45 to transform them into FHIR resources, and once processed, forwards them to the Data Loader 43, which persists the data in the appropriate data store in the SPHERE℠.

Some of the data processed through the Extract, Transform and Loading (ETL) 44 may not easily be recognized and transformed into FHIR resources. These ETL exceptions may include images of text (e.g., PDF), non-text images (e.g., photos), voice recordings (e.g., messages, podcasts). Data extracted from text images are transformed into FHIR resources by a Data Extraction service 47. An Image Processing service will transform non-text image data into FHIR resources 48, and a Natural Language Processing service (NLP) will attempt to transform voice recordings into FHIR text resources 49. Content effectively recognized and transformed by these services are transmitted directly to the Data Loader 43. Content whose structure is not recognized and transformed by these services are transmitted to a semi-automated reviewer 50 that uses AI-based analysis, and human viewing to recognize the content and transform it into FHIR resources, which are then sent to the Data Loader 43, which persists the data in the appropriate SPHERE℠ data store.

### Example Collaboration Channel

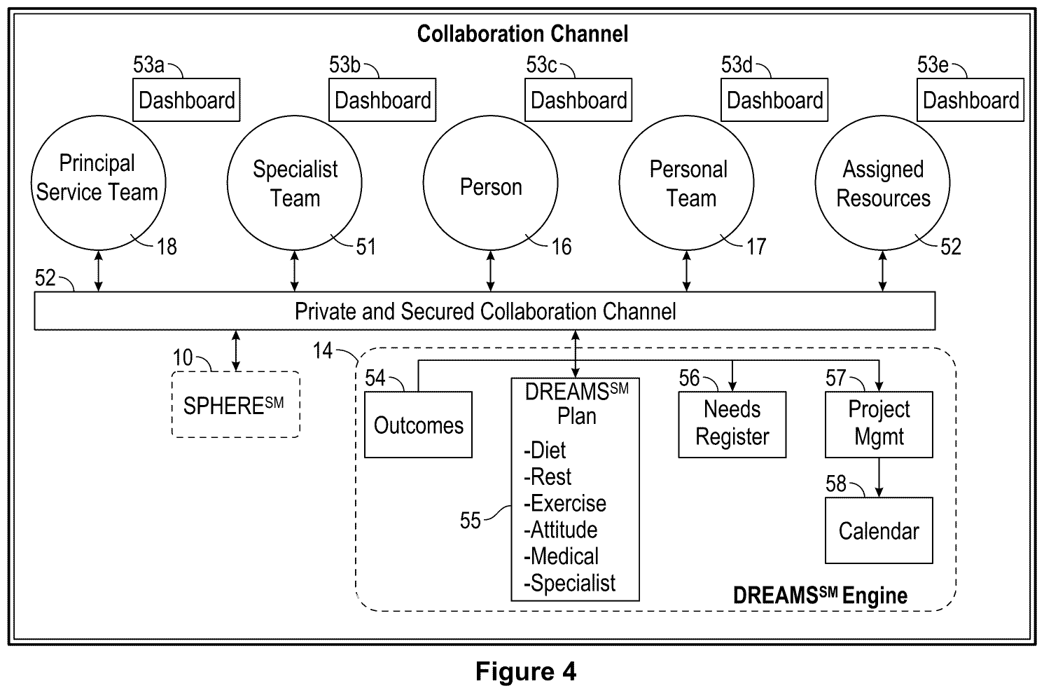

As illustrated in FIG. 4, the Collaboration Channel 13 serves as the backbone of the collaborative platform disclosed herein, providing an innovative, dynamic, multimedia communications channel that is continuously available to facilitate discussion, resource sharing, DREAMS™ planning, and assessment for optimized health, wellness, conditioning, fitness, and performance management and improvement. Collaboration Channel services include private and secured messaging, video-conferencing, and content upload and download. Secured recording services are available for review and archival. When exchange among Collaboration Channel users is warranted, any appropriately authorized user is able to initiate communications and content sharing with any other appropriately authorized user.

The Collaboration Channel is the primary communication facility supporting the Person 16, the Principal Service team 18, Specialist team 51, Personal team 17 and other Assigned Resources assigned specifically to support the Person's care 52 (e.g., pharmacy, medical device service, nutrition service), each of whom is able to quickly review summary data using a role-specific dashboard 53a-e view of the health and progress toward achieving the goals set forth in the DREAMS™ Plan for each person for whom access has been given. The dashboards are designed for two-way coordination of care. One way is for feedback of a person's DREAMS™ Plan outcomes to be reported to the Principal Service team, Specialist team, Personal team, or Assigned Resource as a static point-in-time report. The other way is for the authorized individual's dashboard to provide the opportunity to reply with inputs to be issued back to the person's dashboard as a highlighted suggestion for improvement. This two-way communication enables real-time collaboration and is a responsive approach to timely intervention for best outcomes.

The Collaboration Channel also can be used to support discussions among individual authorized team members. For example, a physician who is a member of the person's Principal Service team and a Specialist might collaborate about best practices for targeting medical procedures covered by value-based care contracts, to determine and optimize ways to coordinate medical care for before and after surgery, with training and education material deployed using the Collaboration Channel 13.

One of the principal outputs enabled by the Collaboration Channel relates to DREAMS™ Plans, including review of outcomes and collaborative agreement on new and updated DREAMS™ Plans. For this purpose, collaborators have convenient access to the data held by SPHERE℠ 10, and the DREAMS™ Plan management and fulfillment services provided by the DREAMS™ Engine 14, including access to outcomes data 54, current DREAMS™ Plan 55, Needs Registry 56, and project management services 57, including the calendar 58. For example, a healthcare provider might use these services as their patient-engagement utility and for collaboratively designing and deploying provider-sponsored education and training targeting specific cohorts or patient populations within a selected population of patients. All communications services and data resource exchanges are protected using the leading privacy and security technology and practices used within the healthcare industry, and consistent with all laws and regulations applicable for any specific platform deployment; e.g., for US deployments, Health Insurance Portability and Accountability Act (HIPAA) Privacy and Security Rules, and for European deployments, the European General Data Protection Regulation (GDPR).

### Example DREAMS™ Engine

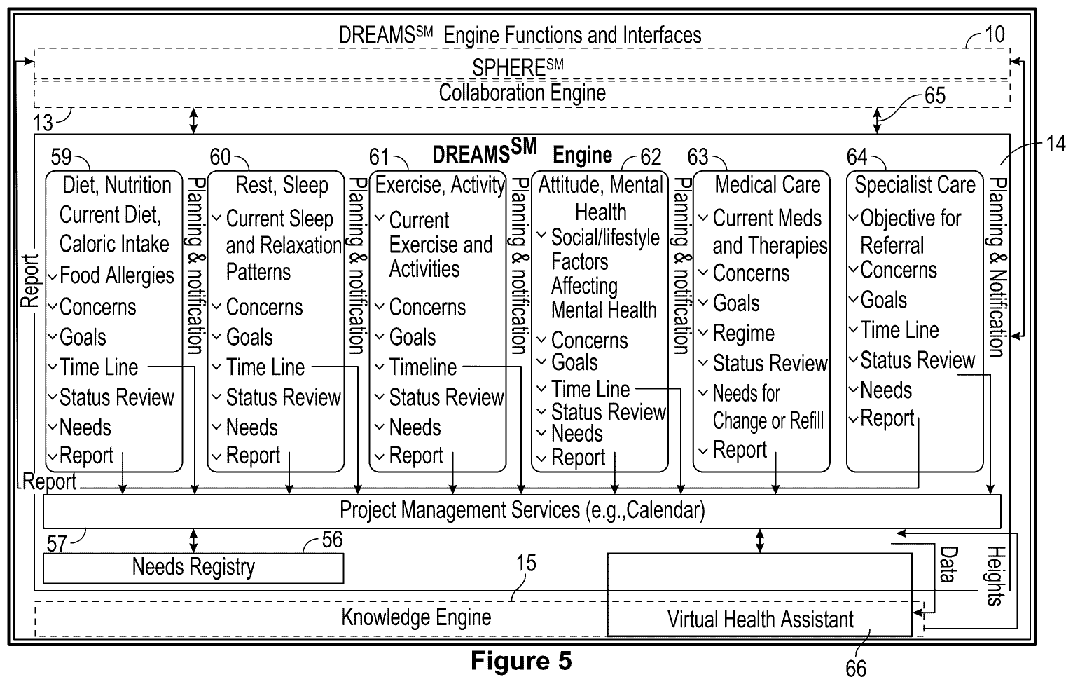

As shown in FIG. 5, at the heart of the platform is the DREAMS™ Engine 14, a set of tools and services for effectively and efficiently establishing, implementing, and coordinating the Person's integrated DREAMS™ Plan, and monitoring, measuring, and incenting outcomes based on that DREAMS™ Plan. This disclosure uses the acronym “DREAMS™” to represent the six interrelated domains that are always addressed in total health and wellness care and management: 1) Diet/nutrition 59, 2) Rest/sleep 60, 3) Exercise/activity 61, 4) Attitude/lifestyle/mental health 62, 5) Medical care 63, and 6) Specialist services 64—and that collectively comprise the DREAMS™ Plan. The DREAMS™ Engine 14 is a structured project-management framework 57 designed as a utility to support the authorized users in managing, incenting, and supporting the fulfillment of the goals set forth in the DREAMS™ Plan, including collaborative establishment of goals 65, with date-specific plans of care in the six health domains. DREAMS™ Engine users use information available from the SPHERE℠ 10, a Virtual Health Assistant 66 powered by the Knowledge Engine 15, project-management tools 57 internal to the DREAMS™ Engine, and a Needs Registry 56 of externally accessed resources uploaded or otherwise shared through the Collaboration Channel 13.

The six health and wellness, interdependent domains encompass categories essential in any holistic, person-centered DREAMS™ Plan for any and all health-and-wellness related endeavors. For example, proper diet should consider such factors as height, weight, vitals, history, gender, and allergies to present to the Personal team. Information relating to these factors is part of the Person's health profile in their SPHERE℠ 10. If the Virtual Health Assistant notes that important information elements of a Person's SPHERE℠ 10 are absent, the DREAMS™ Engine 14 will push to the Person (via the Collaboration Channel) a request to populate the information in their SPHERE℠. For example, if current weight is not noted, the DREAMS™ Engine reminds the Person and Personal team to populate the weight field. Given that multiple members of the Principal Service team 18 and Specialist team 51 may recommend individual care plans, the DREAMS™ Engine implements a step-by-step process to support the collaborators in accessing, reviewing, and evaluating these disparate care plans in order to agree upon one consolidated DREAMS™ Plan across the six health domains. For example, a specialist without visibility into a Person's full SPHERE℠ and care plans prescribed by other providers, might recommend exercises and activities that other providers might consider unproductive or even detrimental to the person (e.g., intensive running may be prohibitive for a person with chronic knee pain).

The internal interoperability among the Collaboration Channel 13, the SPHERE℠ 10 (which is where electronic care plans from all the disparate providers and specialists providing care to the person are stored), and the Knowledge Engine 15 allows authorized users to seamlessly collaborate and create a comprehensive person-centered DREAMS™ Plan. Within the DREAMS™ Engine project-management services 57 structure, each of the six DREAMS™ domains is designed to be managed as a separate “project,”, and all information and collaboration relating to the full extent of that domain is managed within that project. For example, the Attitude/lifestyle/mental health domain includes lifestyle choices (smoking, drinking), mental state, energy level, and all other factors affecting Attitude. So domain by domain, each domain may have different users assigned to collaborate as a separate “project.” Only assigned users can be part of the ongoing management and monitoring of that part of the DREAMS™ Plan.

Once goals for all six domains of the DREAMS™ Plan are established based on inputs from the disparate care plans across all Principal Service team, Specialist team members, and Assigned Resources, and collaborated on, approved, and accepted by the Person and their Personal team, the DREAMS™ Engine can be used to view daily, weekly, and monthly needs and to determine how the needs get fulfilled. Needs are visible from within the calendar, including assigned responsibilities and yet-to-be-assigned needs, allowing users to accept responsibility for filling open needs. For example, if diabetic meals for dinner are prescribed in the DREAMS™ Plan, this need will appear on the Person's calendar as a daily need. A member of the Personal team, such as a family member can mark this need as an item they will provide, and the assignment will be recorded in the calendar. The DREAMS™ Engine enables each Person to configure calendar notifications and reminders as they prefer. For example, the Person might configure the calendar to request confirmation that an assigned meal delivery task was completed at the specified time. Also, if desired, the Person can use the DREAMS™ Engine to capture a photo of the meal and record calorie and portion can be recorded for that meal. A weekly summary is recorded for the Diet/nutrition domain. Similar processes are performed for each domain to keep the Personal team engaged in supporting and fulfilling the DREAMS™ Plan.

The DREAMS™ Engine's care planning functions also include goal setting. An example would be daily caloric intake or salt intake. An activity metric might include the number of calories burned and/or the number of days walking or swimming. The detailed DREAMS™ Plan created through the DREAMS™ Engine's project-management services uses these goals to design the proper fulfillment of each category. As alluded to previously, daily notices go to Persons and Personal team members assigned to each task, reminding them of tasks in advance and following up when tasks are assumed completed. The DREAMS™ Engine detects when information is provided for a task, and if no response has been recorded, generates a reminder via the Collaboration Channel along with an option to provide a response. Any response is recorded by pushing it and its metadata into the SPHERE℠ 10 as “person-generated data,” thus capturing provenance. On a daily, weekly, or monthly basis, activities needed to be performed by the Person can be recorded by the DREAMS™ Engine, such as recording vitals or completing specified physical activities. For example, the DREAMS™ Plan may be set up to have (with prompt, if needed) the person record their blood pressure twice each day, or to record their weight each morning and evening.

At the end of each week, or within a time frame established in the DREAMS™ Plan, the DREAMS™ Engine generates and presents a DREAMS™ summary. The summary can be customized by the Person or any member of the collective support team having been assigned rights to do so. For example, if authorized for their role, a Personal team member can review all meals, a summary of calories by day and week, and exercise activity by day and week. The summary presents a review of level of compliance and highlights areas needing improved compliance. Furthermore, it can compare results against the DREAMS™ Plan to see how effective DREAMS™ planning and fulfillment has been. An example would be a summary showing exceptionally high blood pressure throughout the week and a trend of weight gain and reports of sleepless nights against a DREAMS™ Plan that expected to see stable or lowered blood pressure, weight loss and adequate sleep. This summary helps the Person and collective support team members to evaluate the week's accomplishments, to hone in on known or unknown issues, and to make necessary adjustments to the DREAMS™ Plan and/or incentives, engaging the full power of the platform services, including the DREAMS™ Engine, the Collaboration Channel, the SPHERE℠, and knowledge generated by the Knowledge Engine.

### Example Knowledge Engine

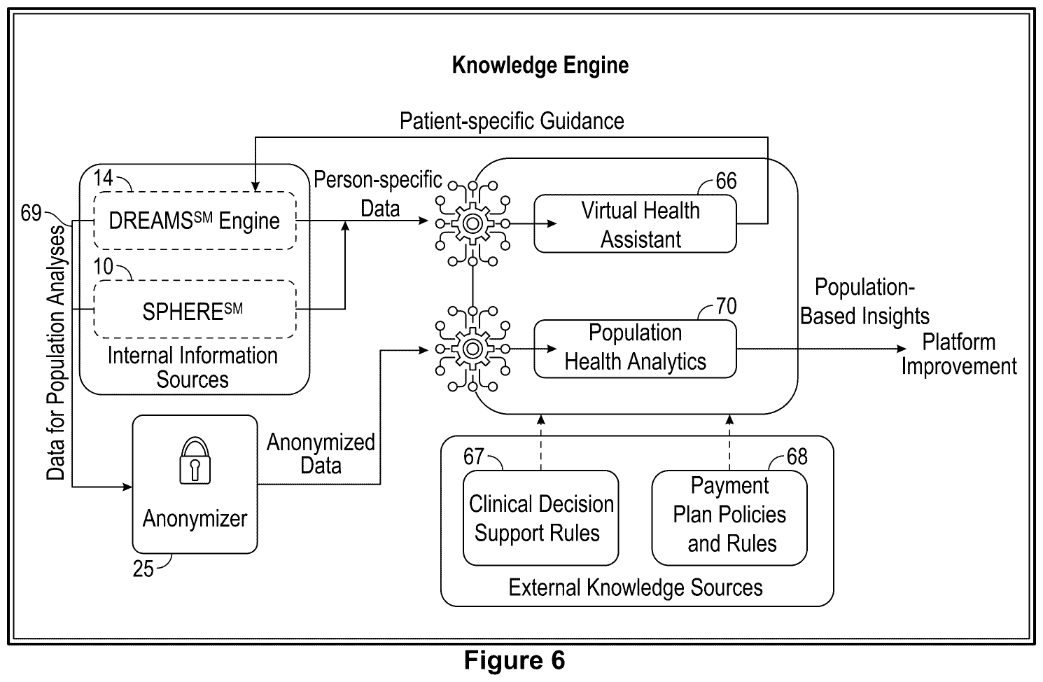

If the DREAMS™ Engine is the heart of the platform, the Knowledge Engine 15 is its brains, using advanced artificial intelligence (AI) and machine learning to analyze both patient-specific data and de-identified population-wide data. As shown in FIG. 6, the Knowledge Engine 15 ingests inputs from both internal and external sources. The DREAMS™ Engine 14 provides patient-specific DREAMS™ Plans, surveys, and progress reports, while SPHERE℠ 10 provides data from its extensive store of person-specific data, including health data, healthcare coverage data, and data collected within the platform. These internal sources provide the Knowledge Engine with both data, as the basis for knowledge, and parameters that may constrain a person-specific analysis, such as PCP-specific limits and payment-plan rules. External sources of knowledge include open-source, prescription-based, and provider-proprietary decision-support rules 67 and payment-plan-specific policy rules 68.

Essentially, the Knowledge Engine is a coded, intelligent engine that can be programmed to scan and detect any condition within the platform and report back as prescribed. Each Person's care will likely be guided and constrained by specific directions and limitations from the Principal Service team, Specialist team, Assigned Resources and from the entities responsible for paying for the person's care. By analyzing these data, the Knowledge Engine enables the Virtual Health Assistant 66 to support the development and fulfillment of person-specific DREAMS™ Plans, including the completeness of information, appropriateness of planned interventions, progress toward DREAMS™ Plan goals, and implications of survey responses. For example, the Knowledge Engine 15 might identify gaps in the Person's SPHERE℠ data or notify the Person's Personal team when a prescribed medication may affect the Person's ability to participate in an activity, or send reminders via the Collaboration Channel 13 to complete activities.

In addition to supporting the Virtual Health Assistant 66, the Knowledge Engine 15 analyzes data from across all instances of the platform 69 to generate Population-Based Analytics 70 with much broader applicability. Person data used in population-based analysis 69 are first anonymized 25 using methods defined by the Health Insurance Portability and Accountability Act (HIPAA) Privacy Rule, or by using other anonymization or pseudonymization methods required or recommended by applicable laws and regulations. The population-based insights 70 generated by the Knowledge Engine are used primarily to enable a learning health environment across all instances of the platform. Both persons and collaboration participants are able to query the Knowledge Engine for both person-specific knowledge for which they are authorized, and general, population-based knowledge. In addition, population-based knowledge is used to continuously improve the power of the platform across all instances.

Accordingly, in some implementations, an electronic, self-learning platform is provided. The platform integrates and orchestrates services and analytics to enable collaborative, integrated planning for health, wellness, fitness, and athletic-performance, change monitoring and measurement, behavior incenting, coaching, supporting, and reporting both internal to the platform and to external authorized recipients.

In some implementations, the platform further comprises a dynamic, goal-directed plan (DREAMS™ Plan) for improving health, wellness, conditioning, fitness, athletic performance, or performance in a profession that demands peak physical and mental capability and execution, by establishing goals, with associated timeline, in the six interrelated domains routinely addressed in total health and wellness management: 1) Diet/nutrition, 2) Rest/sleep, 3) Exercise/activity, 4) Attitude/lifestyle/mental health, 5) Medical care, and 6) Specialist services (DREAMS™).

In some implementations, the platform further comprises an always-on, private Collaboration Channel that enables sharing and exchange of information, impressions, multimedia content, and feedback, aimed toward integrating and managing the DREAMS™ Plan among the individual who is the primary focus of the platform (person), the family and friends who provide ongoing support to the Person in meeting the goals set forth in the DREAMS™ Plan (Personal team), the team of health and wellness professionals who provide principal support to the person (Principal Service team), the team of specialists supporting the Person (Specialist team), and individuals who fulfill individual needs identified in the DREAMS™ Plan (Assigned Resources).

In some implementations, the platform further comprises an integrated, dynamic DREAMS™ Engine project-management and fulfillment service that integrates multiple, disparate domain-specific, prescribed plans and plan-of-care algorithms assigned to the person into a single DREAMS™ Plan, including services to support, manage, and incent fulfillment of the DREAMS™ Plan within the interrelated domains, and for measuring and improving outcomes based on the DREAMS™ Plan. This project management and fulfillment service also supports scheduling tasks, events and activities with calendar-based planning and management tools, as well as managing task dependencies and task interdependencies, assigning of task, event and activity owners, and tracking of progress and completion of tasks, events and activities to support notification of appropriate users.

In some implementations, the platform further comprises a dynamic Secure Personal Health Electronic Resource Exchange (SPHERE℠) that integrates the Person's health-system records with any other digital content related to the person's health, wellness, conditioning, fitness, athletic performance, or professional physical, mental or other performance gathered from external sources, including care providers, athletic trainers, coaches, labs, specialists, and other individuals and organizations, along with genomic data, social determinants of health (SDOH), device data, financial, cost and/or claims data, and other Person-generated data provided through uploads, responses to surveys generated within the platform, and outcomes feedback from the execution of the integrated DREAMS™ Plan.

In some implementations, the platform further comprises a robust rules-based decision support engine that uses programmed and learned rules based on applicable DREAMS™ Plans, SPHERE℠, and other system data, in order to dynamically trigger a variety of activities such as generating system alerts and providing notifications regarding missing information; identifying changes to DREAMS™ Plans and/or SPHERE℠ data and/or other system data, notifying appropriate users when tasks, events and activities are completed, on pace with or falling behind expected progress, or that need to be completed; alerting the Person and other authorized users of changes in team composition and/or role/access capabilities; and/or suggesting new activities or changes to activities based on the rules engine and updated data.

In some implementations, the platform further comprises a content-capture, integration, and management architecture and support services that enable receipt, recognition, transformation, metadata tagging, and persistence of any structured or unstructured digital content.

In some implementations, the platform further comprises a robust Virtual Health Assistant that consumes person-specific data to continuously analyze current state, identifies data omissions and potential adverse events, and generates additional insights that may be useful in modifying and/or fulfilling the DREAMS™ Plan, and provides feedback to the Person, Personal team, and platform services.

In some implementations, the platform further comprises a robust Knowledge Engine that consumes and analyzes anonymized data across persons within any single instance of the platform, and across all instances of the platform, to acquire new, population-based knowledge, enabling a continuously learning environment accessible to all platform instances and users.

In some implementations, the platform further comprises metrics, methods, and technologies for measuring progress toward meeting DREAMS™ Plan goals, and a feedback mechanism tailored to motivate the Person to which the DREAMS™ Plan pertains, to support meeting goals and positively modifying daily living behaviors and activities toward better outcomes as defined in the applicable DREAMS™ Plan.

In some implementations, the platform further comprises a roles and rights management services that enable the Person to whom the DREAMS™ Plan pertains (and that person's proxy) to assign roles, with specific role-based associated access to data and services, to each member of the Person's extended team.

In some implementations, the platform further comprises, for each role defined and implemented within any given instance of the platform (e.g., Principal Service team, Specialist team Services Provider, Personal team, Person), a customized dashboard that at any given point in time, provides a summary snapshot of the current state and progress for each person, in accordance with the rights assigned by each person, in order to help manage that person's care and/or wellness.

In some implementations, the platform includes architectural features and functional services designed to protect against accidental and intentional security threats to the confidentiality, accuracy, and availability of the data and services.

In some implementations, the platform includes architectural features and functional services to enable an individual Person or their assigned proxy to assign individual roles and rights to each individual on their support team.

In some implementations, the platform is used within a Person's extended context for the purpose of facilitating collaborative, informed, management and improvement of that individual's health, wellness, fitness, conditioning, athletic and mental performance, or high performance in a profession that demands peak physical and mental capability and execution.

According to some implementations, a method is provided for health and wellness management. The method includes generating a health and wellness plan for a Person that includes (i) a diet (or nutrition) plan, (ii) a rest (or sleep) plan, (iii) an exercise (or activity) plan, (iv) (an attitude or) a mental health plan, (v) a medical care plan, and (vi) specialist services, based on a repository of health and wellness data that is specific to the Person.

In some implementations, the health and wellness plan (sometimes called a DREAMS™ plan) is an aggregation of patient centered inputs from various sources of health information on a patient (sometimes called a person) from disparate clinical record resources, a patient's genomic data, a patient's socially determined data, a patient's self-determined health status, a patient's data that is self-generated, such as self-reported diet, rest, exercise, attitude/lifestyle, medications and specialties of all varieties. This aggregated patient data is made accessible to a collaboration of authorized individuals, clinical and non-clinical who are assigned certain roles and rights to engage electronically in the patient care planning to a specific plan for the target patient. These collaborators can review the patient health information and using this information seek outside medical and other research to help support a determination of best integrated plans of care with the ability to assess each domain of plan with consideration of patient specific profile and any contraindication from other domains or otherwise useful information from outside sources to pin point whole person care plans or precision medicine-based plans.

Examples of a health and wellness plan are described herein, according to some implementations. A DREAMS™ plan contains a set of goals and a set of activities, with each activity tied to one or more of the goals. The goals are associated with one or more DREAMS™ domains; there are six discrete domains each that relate to specific care components. The components are aggregated as FHIR care plan resources. These domains are each constructed per FHIR care plan specifications. From an end-user perspective, the six individual domains are created using system tools. These tools enable goals to be set and interdependencies to be considered (e.g., diet relative to exercise). Following is an example of a DREAMS™ plan:
Diet
   Goal: Lose 40 pounds by Dec. 31, 2022
      Activity: Adhere to 2000 calorie diabetic diet
      Activity: Limit in between meal snacks to only healthy vegetables in reasonable quantities
      Activity: Walk a minimum of 1 mile per day
      Activity: Consult nutritionist for further diet advice
      Activity: Measure and record weight at least every 3 days
   Goal: Reduce Hemoglobin A1C level to below 7.0 by Sep. 30, 2022
      Activity: Activity: Adhere to 2000 calorie diabetic diet
      Activity: Take one Metformin 500 mg tablet twice a day at breakfast and dinner
Rest
   Goal: Sleep at least 7 hours per night by Aug. 31, 2022
      Activity: Wear device that tracks and monitors sleep quality and habits
      Activity: Go to bed prior to 12 midnight every night
   Goal: Keep AHI (Apnea-hypopnea index) to less than 5.0 by Aug. 31, 2022
      Activity: Wear device that tracks and monitors sleep quality and habits
      Activity: Self monitor nightly AHI and follow-up with primary care provider is AHI is above 5 for 3 or more nights in any 7-day period
Exercise
   Goal: Exercise at least 30 minutes per day, and increase duration by Sep. 30, 2022
      Activity Start walking (either outside or on a treadmill) a minimum of 1 mile per day. Increase the walking distance to at least 2 miles a day by Sep. 30, 2022
Attitude
   Goal: Develop a more positive attitude regarding self and ability to lose weight and control diabetes
      Activity: Keep an ongoing log journal of both positive and negative thoughts related to self and ability to lose weight and diabetes
      Activity: Take attitude/lifestyle survey every two weeks
Medicine
   Goal: Adhere to prescribed medication schedule
      Activity: Take one Metformin 500 mg tablet twice a day at breakfast and dinner
      Activity: Take two Ibuprofen 200 mg tablets every 6 hours as needed for right shoulder pain
Specialist
   Goal: Make appointments to see recommended specialists by Aug. 31, 2022
      Activity: Consult nutritionist for further diet advice
      Activity: Consult orthopedic surgeon for chronic right shoulder pain related to previous injury

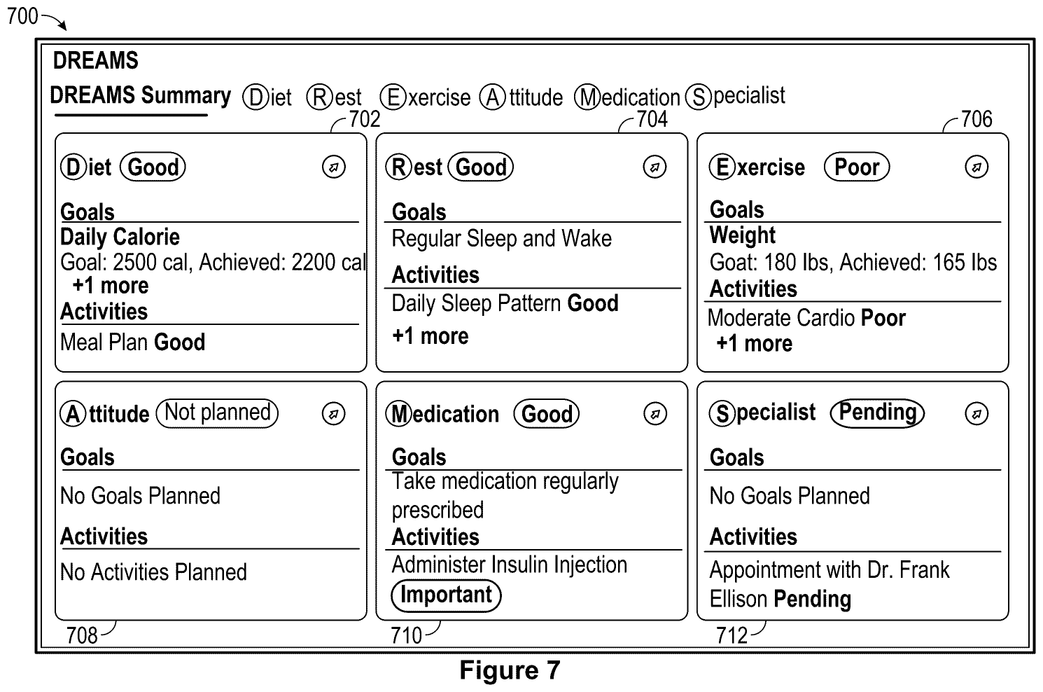

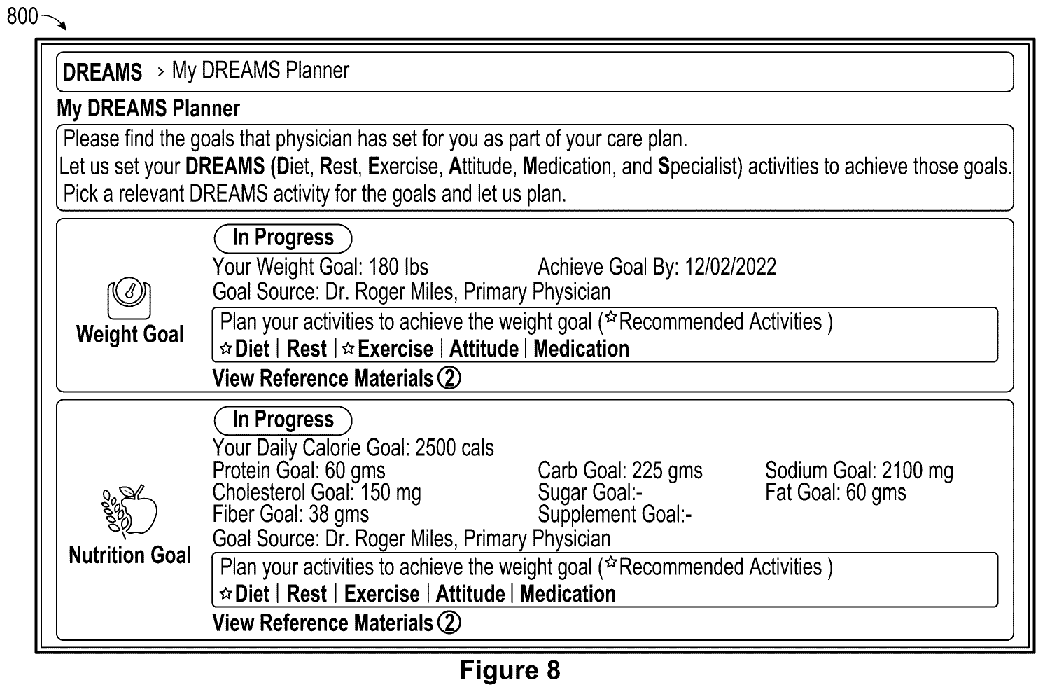

The health and wellness plan may be implemented using different formats. While the DREAMS™ Plan data are exchanged as FHIR resources over RESTful interfaces in order to allow them to be shared in a standard format, that format may not be suitable for non-technical users to interact with. Therefore, in some implementations, the data contained in those FHIR resources is displayed to the end users in a specific format in order to make the DREAMS™ Plan easier for end users to view, assess progress and update. FIG. 7 shows an example visualization 700 of a DREAMS™ Plan summary, according to some implementations. The summary includes details for diet 702, rest 704, exercise 706, attitude 708, medication 710 and specialist 712, according to some implementations. FIG. 8 shows an example visualization 800 of a DREAMS™ planner, according to some implementations.

A DREAMS™ Plan may only have some elements (e.g., domains) activated, and not all elements may be required. For example, if a Person's weight is within a normal range and the Person has no health issues or wellness goals related to diet, there may not be any diet goals or activities for that Person. Data fields may be represented in a DREAMS™ Plan as FHIR Care Plan resource, but not all fields may be populated for each Person.

An example of how the SPHERE℠ is used to generate a DREAMS™ plan is described herein, according to some implementations. A DREAMS™ Plan may use electronically imported care plans from multiple provider EHRs and stored in SPHERE℠ The DREAMS™ engine enables authorized users to retrieve the Person's health profile information from SPHERE℠. This information is used for constructing the DREAMS™ Plan. SPHERE℠ may contain care plan resources from providers and specialists and may identify provider or specialist and other metadata that as origins (provenance) for the information. The DREAMS™ plan may include links to these source (provider) care plans and indicate whether information is based on, replaces, or is part of, relevant other information (or an original source of information). Additionally, the SPHERE℠ may be used as a source of exemplar whole-person health information and profiles to support effective determination of goals for each domain. An example is a patient exercise plan. A Person's medical care plan's problem list may state a current problem with neuropathy on both feet with great pain walking. This information is used by clinical and personal care teams to select exercise other than walking. It may also support the decision to see a specialist for the health condition. This might include a chiropractor who specializes in therapy for resolving neuropathy. Another example where the SPHERE℠ is used to generate elements of the DREAMS™ plan might be the clinical documentation of food allergies or gluten intolerance. Having access to this information at the point of collaborating on diet needs would help the team to avoid certain diet considerations.

Different algorithms may be applied in generating a DREAMS™ plan, including algorithms from nationally recognized societies and organizations (e.g., the American Heart Association Blood Pressure Management Algorithm, the American Diabetes Association Pharmacologic approaches to glycemic treatment: Standards of Medical Care algorithm), algorithms developed or utilized by client organizations, such as a client specific algorithm to manage dementia, and/or a client developed or third party algorithm used by a client to manage patient weight loss. The platform may be designed for clinical teams to use these standards and embed rules and conditions based on these algorithms that can be stated as a means of setting goals using templates and extended across all the DREAMS™ domains as a complete protocol. This template may then be assigned to patient cohorts with health profiles that are meant to be addressed with the algorithms.

Some implementations use a template, for the DREAMS™ plan, that is a predefined electronic, partially or completely finalized document which then can be edited and customized for use with an individual patient or other user. A template may be a predefined set of goals and/or activities with default values to address one or more specific issues that have been pre-approved to be used by the organization, (e.g., a Principal Service team of an Accountable Care Organization) implementing the platform for a specific cohort of patients based on stated chronic diseases and/or health and/or wellness objectives to support more effective population health initiatives. The user can use the template for the goals, activities and default values without making any changes, or can modify any or all of the goals, activities and default values in the template to meet individual user needs. The use of templates helps standardize care across an organization as well as save user time entering and configuring goals, activities and their default values. An example template is the FHIR CarePlan (available online at hl7.org/fhir/careplan.html).

For cleansing raw data, various methods may be used. Some implementations use security technology to ensure that any incoming data are safe. For example, the system may check the data to make sure the data does not contain any embedded executable code, including executable SQL. After this step, data is then processed using extract, transform, load (ETL) technology to safely extract relevant information, transform the data into structured (FHIR) resources that the platform can consume, and loaded into SPHERE℠ Persons, and their authorized DREAMS™ team members, may be notified that new data are available. Some implementations may use surveys that are structured electronic documents or forms whose content are predefined by a provider; for example, the forms may include specific questions in human-understandable format. Responses to survey questions may be captured directly from a patient via access to internet enabled devices into a format (e.g., FHIR) that is consumable by SPHERE℠. In some implementations, a back-end of the system may be partitioned into several trust zones, and operated under a zero-trust model, where exchanges between zones are structured and examined to assure that they are both safe and presented using appropriate, consumable formats.

The method also includes selecting a plurality of individuals (e.g., family and friends, healthcare and wellness professionals, specialists, other resource providers) to form a support network for the person based on the health and wellness plan. The method also includes assigning roles, responsibilities and role-based access rights for the plurality of individuals in the support network, based on the health and wellness plan.

Examples of how roles may permit or restrict access to specific data and/or services are described herein, according to some implementations. Role-based access control enables individual users (and processes) to access specific functions and resources. Typically, role-based access control is implemented based on roles that are predefined by system administration (e.g., admin users can access {broadly-defined set of functions and resources}, non-privileged users may access {limited set of functions and resources}, and so on.) Non-privileged users (e.g., Persons) then can assign these pre-defined roles to specific users (e.g., Personal team), enabling those users to perform a specific set of functions (e.g., read, modify) with respect to the Person's personal resources.

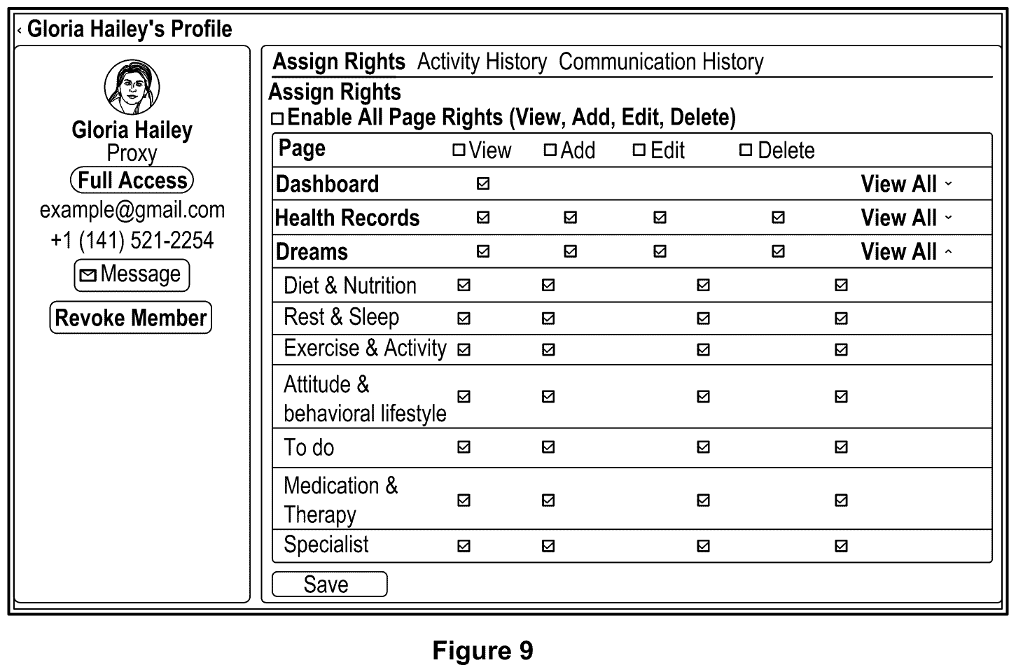

In addition to providing Persons with a menu of pre-defined roles, the system may also provide the capability for Persons to define their own rules. In some implementations, not all users may define roles-just primary users (e.g., Persons (or proxies). The system may provide a Person (or a proxy) a toolbox that includes a set of pre-defined roles, along with a subset of individual rights that may be combined to create customized roles that are subsets of the pre-defined roles made available to the Persons. For example, the system may enable all Person's to assign a {create-read-write} role for anyone in the Person's DREAMS™ Team. But the Person may want to give specific members only {read} access. The system may enable the Person to assign this limited role to specific individuals. The system may enforce both pre-defined and customized role-based accesses through a rights-based enforcement mechanism. FIG. 9 shows an example user interface for the configuration of role-based access controls by Persons, according to some implementations.

In some implementations, the system may assign user roles, including specifying and permitting use of tools and defining access to resources, such as SPHERE resources. An example of this is a specialist, such as a dietician, needing only to see certain patient information and not all information. The dietician could be permitted access to all diet goals and plans, all health information related to diet restrictions or allergies, medications and exercise since these are what a dietician most often needs to make a whole-person assessment and plan for diet. All other health information beyond this may be restricted. If the dietician needed more information, they would need authorization from the Person. All system-specified and Person-specified roles are enforced by the system.

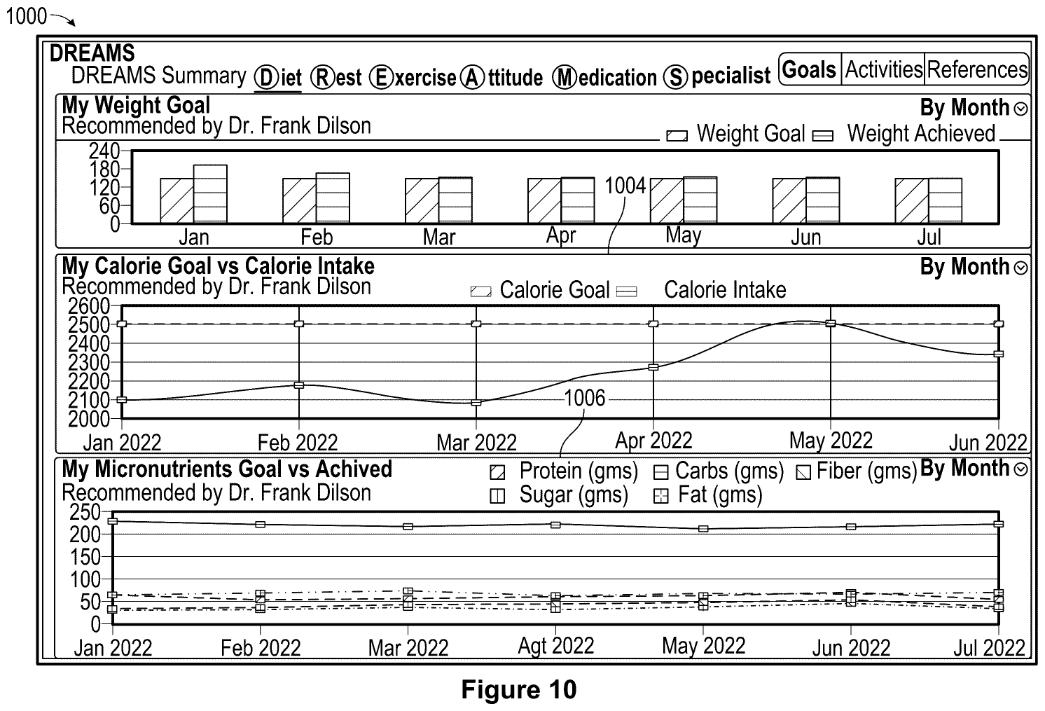

The method also includes reporting and updating the health and wellness plan for the Person, based on tracking health of the person. For example, when a goal or activity includes a specific duration or timeframe for meeting that goal, the system may have a general set of default values that it will use to measure progress on meeting the goal as specified in the DREAMS™ Plan (for instance, 15% progress towards achieving the goal or activity from the baseline value when 25% of the duration/timeframe has passed, 40% progress towards achieving the goal or activity from the baseline value when 50% of the duration/timeframe has passed, 70% progress towards achieving the goal or activity from the baseline value when 75% of the duration/timeframe has passed, and 100% progress towards achieving the goal or activity from the baseline value when 100% of the duration/timeframe has passed.) The default values set in the system may be customized or modified to meet each Person's needs or set for populations of certain disease cohorts, for tracking progress to each goal. When a DREAMS™ Plan goal or activity is measured more qualitatively than quantitatively, the system may then prompt the relevant user to assess and enter their progress on meeting that goal or requirement. Some qualitative goals or activities may be met based on the presence of some document or piece of data such as a form or electronic survey created with the system tools previously defined, (for instance, complete an attitude survey monthly). Surveys may be represented in SPHERE℠ as FHIR Questionnaire resources (e.g., available online at hl7.org/fhir/questionnaire.html), enabling the system to store responses that are interoperable with other health data stored in SPHERE℠. Because the questionnaire responses are integrated with a person's record in SPHERE℠, the system may then automatically determine when the user has completed the required document or entered the required piece of data without having to query the user. The user's progress may then be summarized on the DREAMS™ Plan summary with an indicator (such as the “Good”, “Poor”, “Pending”, and “Not Planned” indicators shown for each of the DREAMS™ domains in FIG. 7), as well as indicators to track progress on specific goals and activities through the use of various types of graphs. FIG. 10 shows example graphs 1000 for tracking specific goals and activities, according to some implementations.

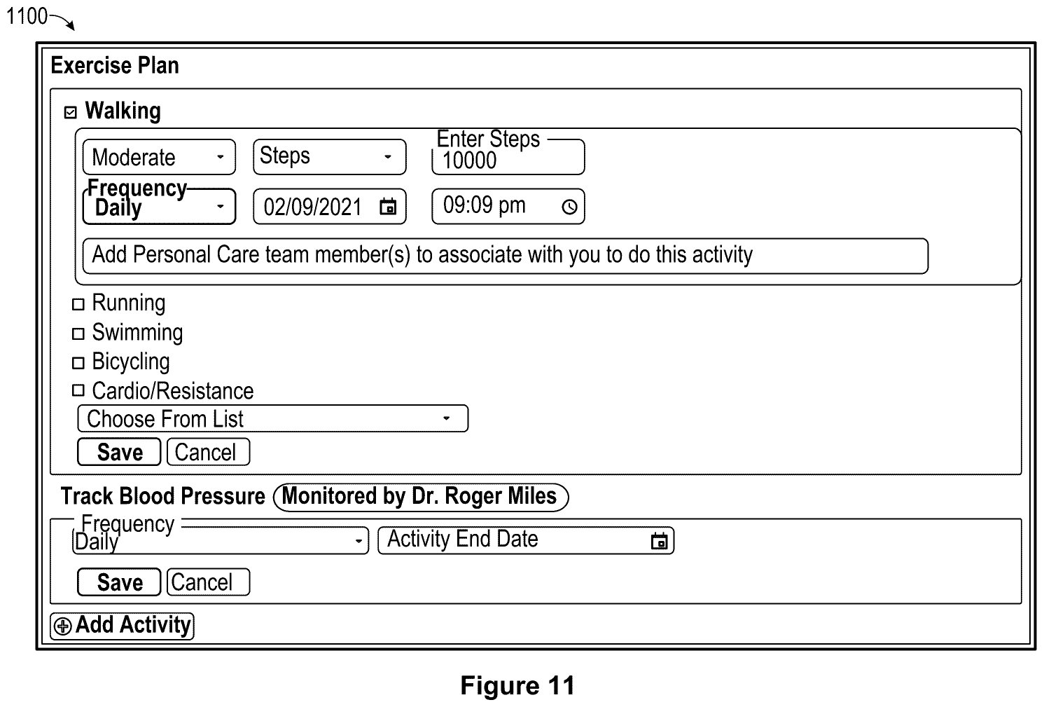

Updates to a DREAMS™ Plan may be performed in a variety of ways, examples of which are described herein, according to some implementations. A DREAMS™ plan may be configured as a dynamic plan that is adaptable based on the ever-changing user needs. Towards that, the system may contain a set of FHIR-based tools that will allow a user to modify DREAMS™ Plan goals and activities when a DREAMS™ Plan needs to be updated. Updates may include updating existing DREAMS™ Plan goals and activities (including the ability to update values, dates, and other parameters associated with those goals and activities), adding new DREAMS™ plan goals and activities, or archiving existing goals and activities. FIG. 11 shows an example user interface 1100 for updating a DREAMS™ Plan, according to some implementations.

Tracking and updating a DREAMS™ Plan may be based on a rule-based decision algorithm. For example, rule-based decision algorithms may be used in tracking DREAMS™ Plan progress and identifying if or when updates need to be made, and for developing a machine-generated assessments of individual users' behaviors (e.g., monitoring and encouraging/rewarding usage, or detecting misuse). Rules-based algorithms may continuously monitor SPHERE℠ to detect when scheduled behaviors have/have not been completed (e.g., completion of surveys, not entering vitals data according to plan or not taking medications as set in the DREAMS™ plan). Roles-based monitoring may be used to build profiles of individual users, which the system may then use to customize services to individual users. Some users may need frequent reminders to complete scheduled activities, while others may perform well with fewer reminders. The platform may enable the aforementioned interaction within the platform among authorized users based on roles, rights and access to the DREAMS™ plan and tools to support collaboration.

The rules-based machine-learning component may not be the only components that encourage an individual's behaviors. The system may also rely on the Person, the DREAMS™ team, and/or clinical care team's review of data to also track DREAMS™ Plan progress and determine if and/or when updates to the DREAMS™ Plan need to be made. This type of monitoring by care teams of the Patient may then use internal communications to alert a patient and others to patient's health status or progress or needed interventions.

In some implementations, the method further includes: generating the repository of health and wellness data for the Person by integrating the person's health-system records with other digital content related to the Person's health, wellness, conditioning, fitness, athletic performance, or professional physical, mental or other performance gathered from external sources, including care providers, athletic trainers, coaches, labs, specialists, and other individuals and organizations, along with genomic data, social determinants of health (SDOH), device data, financial, cost and/or claims data, and other person-generated data provided through uploads, responses to surveys generated, and outcomes feedback obtained during execution of the health and wellness plan. In some implementations, the repository is aggregated from multiple sources, including healthcare encounters, fitness training, wellness training, athletic coaching, strength and conditioning training, and other health-and-wellness-related encounters within and outside the healthcare system (including financial, cost and claims data), along with assorted person-specific content generated and/or uploaded through the use of platform services.

Examples of data integration and/or aggregation are described herein, according to some implementations. Because data may be captured and persisted as RESTful FHIR resources, they are easily integrated, monitored, and/or retrieved; this is supported by a micro-service-based architecture. Persons may retrieve their medical data as FHIR resources from multiple providers. Some implementations persist these data within SPHERE℠ (e.g., as FHIR resources). Further, some implementations allow Persons to supplement (or automatically update) their SPHERE℠ health record with data collected through surveys (e.g., social determinants of health (SDOH), performance metrics, compliance data), uploaded documents, and internet connected devices (or manually entered data where no internet connected devices are used, such as rest or sleep data where number of hours sleep is entered), and to persist all of these data as FHIR resources within SPHERE℠. Because data persist consistent with FHIR resource standards, providers may retrieve this vast store of valuable health-related data as FHIR resources, easily integrated into their patients' existing records. Some implementations provide a write-back FHIR application programming interface (API) that will enable providers and patients to upload new health-related information (e.g., updated addresses, health plan information, lab results) as FHIR resources, and to integrate these data into the patients' electronic health records (EHR). The API allows persons to write back requested health data to their providers for more appropriate, personalized, whole-person care. Some implementations use Health Level Seven (HL7) FHIR standards for data file format to collect and persist data uniformly, and to exchange data over standardized, RESTful APIs. In some implementations, aggregation and/or integration may be achieved through consistent use of FHIR resources for data representation and exchange.

Feedback from execution of the health plan may be used to update the SPHERE℠ in different ways, according to some implementations. For example, execution results are captured as updates to FHIR CarePlan resources stored in SPHERE℠. Once a Person is set up with a DREAMS™ Plan they may begin daily interaction with the plan. For example, the daily plan may be presented to the patient indicating they need to take medication at a certain time, need to record their blood pressure at a certain time, need to chart their meal at a certain time, need to record their exercise at a certain time to comply with the plan. As these events are completed, the patient may report they have been completed and enter the value into the defined area where data is to be entered (or captured electronically and imported and reflected) in their DREAMS™ Plan as an update to the plan and the data is stored in SPHERE℠ as a FHIR resource in profile. The Person-generated health data points are updated in the repository in the predefined fields and become part of the Person's whole person profile in SPHERE℠ where all DREAMS™ components are defined as part of the FHIR Care Plan resource format.

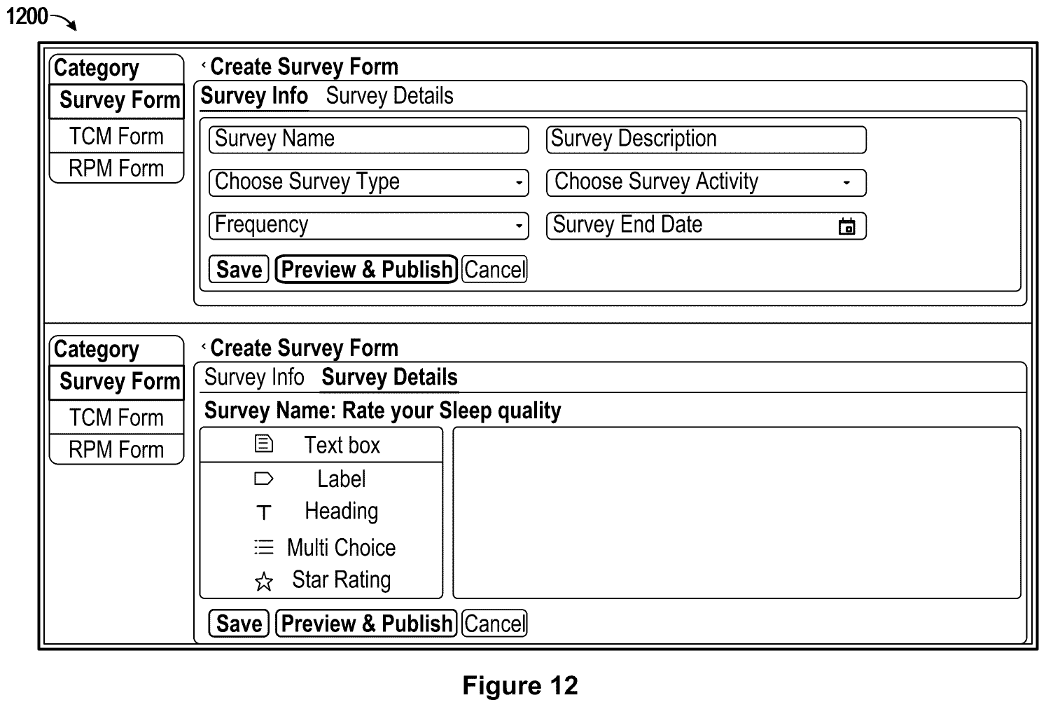

Some implementations use surveys that are tools that a Person or a member of their personal support or clinical care teams is asked to fill out to provide relevant information regarding that user at that point in time. Examples of surveys include a pain scale survey, a mental health survey, a diet survey, an exercise survey, or a social determinants of health survey. The surveys can either be surveys that have already been developed by a nationally recognized organization or society or could be surveys developed by a client organization. The surveys may be created as FHIR Questionnaire resources (e.g., questionnaire available online at hl7.org/fhir/questionnaire.html) and presented in a survey format through the UI/UX. Data entered into the survey form may be captured as FHIR questionnaire responses and stored in a Person's SPHERE℠ health record. Some survey responses may trigger responses from the rules-engine, and possible feedback to the Personal team and/or relevant provider. FIG. 12 shows an example user interface 1200 for creating surveys, according to some implementations.

In some implementations, execution of a Person's DREAMS™ Plan is the Person's ongoing performance of the activities defined in that Person's DREAMS™ Plan, assessment of that performance in meeting the DREAMS™ Plan goals and activities within the specified time period, and capturing results in the DREAMS™ Plan (persisted in SPHERE℠). Depending on the content of the DREAMS™ Plan, reported results may trigger a rules-engine response, such as an update to the Personal team or a warning to the relevant provider or specialist.

In some implementations, the method further includes providing an always-on, private collaboration channel that enables sharing and exchange of content (e.g., information, impressions, multimedia content, and feedback), for integrating and managing the health and wellness plan among the person and the plurality of individuals in the support network. In some implementations, the collaboration channel is multi-media capable and supports a team-approach to defining specific goals toward improvement of health and wellness within specified timelines. In some implementations, the collaboration channel provides processes and services for sharing information, experience, and content among users, including secured email, messaging, alerts, notifications, reminders, secured upload and download and real-time audiovisual collaboration. In some implementations, the collaboration channel enables uploading of any digital content from any internet-connected device, with content-recognition and transformation tools available to analyze, transform, persist, and manage the content in a standardized syntactical and semantic format for facilitated retrieval and use.

In some implementations, the collaboration channel is always on for asynchronous communications (e.g., intra-application secure electronic communications, including attachments), uploading/downloading of assigned/permitted library content, and scheduling of synchronous events. For example, if a Personal team member wants to schedule a video-conference, the scheduling can be done at any time. The secure and private video-conferencing capability also is available 24/7, which is particularly useful across time zones.

Data may be uploaded in a variety of ways. For example, content (including paper scans, text, and multi-media) may be uploaded through message attachments and responses to posted queries. Also, personal health-related device data may be uploaded through proprietary and open APIs required by the individual devices. Data may be captured by the system using any electronic device enabling access to the platform with proper authorization and rights to use the upload capability. When a user is properly credentialed to do so, they may click an upload feature and select a file they want to upload. The file is then uploaded into the platform to a specific predefined location. Data may also be captured by entering into predefined fields, such as “Enter Weight” that is input by patient and recorded in the patient's profile under “Weight” with the metadata tags indicating time, date and an identity of device used for the upload. Various standardized syntactical and semantic format may be used to analyze, transform, persist, and manage content. For example, FHIR resource standards, including the observation resource available online at hl7.org/fhir/observation.html, media resource available online at hl7.org/fhir/media.html, and/or questionnaire resource available online at hl7.org/fhir/questionnaire.html may be used.

In some implementations, the method further includes: using an artificial intelligence (AI)-based knowledge engine to: (i) continuously receive data for the person, analyze a current state, identify data omissions and potential adverse events, and (ii) generate insights for modifying and/or fulfilling the health and wellness plan based on the current state and identified data omissions and potential adverse events; and providing feedback to the person and the plurality of individuals, based on the generated insights.

Examples of self-learning capabilities, such as algorithms used, type of data used for learning, adjustments made to relevant data stores or decision processes, are described herein, according to some implementations. In some implementations, self-learning applies with respect to both the individual and the platform itself. A rules-engine monitors person-specific (i.e., identified) data to assure that data are complete and to develop (“learn”) a profile of each person to personalize responses and intensity of intervention. For example, if a person frequently asks for clarification, some implementations may simplify content or pre-answer anticipated questions and provides more helpful hints. If a Person consistently updates their daily monitored data, as specified in their DREAMS™ Plan, some implementations may not provide as many reminders as to that person when compared to an inconsistent user. Also, a provider may request that a Person's condition and interactions be more carefully monitored, and some implementations may do so on an individual basis.

In addition, some implementations may use de-identified data stores to learn population trends and needs for platform improvements. The ongoing utilization of machine learning against de-identified data stores gives the user organization the ability to discover deeper insights not otherwise available in other platforms, due to the breadth of data stored in a SPHERE℠ profile. The aggregation and inclusion of the volumes and diversity of patient generated health data, along with data on patient compliance, is a complexity that cannot be handled in conventional platforms.

As used herein, data for the Person refers to any information that the system (sometimes called the platform) receives for its users being person to whom a DREAMS™ Plan pertains, care team members or any authorized user engaged in use of the platform. A current state refers to a current state of the Person to whom a DREAMS™ Plan refers to within a context. Assessing a Person's current state may include identifying what data are held, what data are needed to provide optimal support. For example, some implementations determine essential data missing (e.g., omissions), a total set of data held for that person. Some implementations determine data metrics at a specific time. The total set of information held for any individual Person at a point in time may define the current state of the Person at that point in time with respect to the total SPHERE℠ record for the individual, and the specific components of the DREAMS™ plan. An example is a Person's weight on a specific date to then relate to the Person's diet goal. This may also include exercise or activity data on a given date to correlate a weight goal to an exercise goal to a diet goal to identify issues with compliance of the plan and progress towards the goal. In some implementations, there is no state machine as such, but transitions between states are driven primarily by the Person, the Person's Personal team, and relevant medical and specialist team working with the Person, and secondarily by the rules-based engine and machine-driven profile of the Person.

In some implementations, data omissions are identified as data that the system expects to be present platform at that point in time but the data is not present. Examples include demographic information, such as birth date or address, zip code where patient works and/or lives, clinical information, such as a recent Hemoglobin A1C level, information from internet of thing devices, such as sleep data from a CPAP machine or exercise information from a smart watch, or uncompleted surveys that the system expects to have already been completed. Some implementations provide a set of tools to set up conditions and rules for a variety of health conditions and vitals that are important to monitor. Using these conditions and rules for an individual Person or a population of persons, the conditions or guidelines may be constantly monitored for status. If blood pressure spikes and Person's weight increases unexpectedly and diet and exercise are omitted for the past week, some implementations generate an alert for a predetermined set of authorized users and the Person. Examples of adverse events include identifying poor quality of sleep that starts when a new medication is prescribed to the user, unexpected weight gain that results when a user is told to discontinue a medication, an increase in knee and back pain, or abnormal heart rate, when the user begins exercising on a treadmill. Data omissions and adverse events may be identified when the system's rules-engine monitors the patient data using system-driven rules and machine-learned knowledge.

In some implementations, the knowledge engine may apply system-defined rules and machine-learning models to users' specific data to generate specific insights and individual profiles. Only deidentified data may be used for population data and platform improvement. Any learning specific to a user may use identified data as well as deidentified data. These machine-driven user profiles may be used not only to improve the health support and generated insights, but also to detect potential misuse of the platform.

Examples of how the system may provide feedback to the Person and the plurality of individuals, based on the generated insights, are described herein according to some implementations. For example, the knowledge engine may generate Person-specific insights by using rules-based monitoring of each Person's identified data and machine-learning knowledge generation to create individual user profiles, as described above. Some implementations may deidentify user data for use in generating population-based insights and needs for potential platform improvements. In some implementations, using deidentified data, artificial intelligence (AI) models are used to identify previously unidentified population-based correlations, associations, and dependencies. Examples may include (i) identifying a previously unrecognized association between taking a medication and an elevated lab result in users with a specific disease that might result in a medication list change for users with that disease and taking that medication, or (ii) identifying a previously unrecognized correlation between eating a certain class of vegetables and improved dementia status that could result in dietary change recommendations for a dementia patient.

In some implementations, the method further includes: analyzing, using an AI-based knowledge engine, anonymized data across a population including the person (e.g., using machine learning to analyze both patient-specific data and de-identified population-wide data), to obtain population-level knowledge; and providing feedback to the person and the plurality of individuals, based on the population-level knowledge. As described above, the knowledge engine may generate insights by using AI models on deidentified system data to identify previously unidentified correlations, associations, and dependencies. Those insights may be applied to specific individual users or general populations. Examples may include (i) identifying a previously unrecognized association between taking a medication and an elevated lab result in users with a specific disease that might result in a medication list change for users with that disease and taking that medication, or (ii) identifying a previously unrecognized correlation between eating a certain class of vegetables and improved dementia status that could result in dietary change recommendations for a dementia patient.

In some implementations, the method further includes: measuring progress towards meeting goals of the health and wellness plan; and providing feedback to motivate the person and modify daily behavior and activities of the person, to meet the goals, based on the measured progress.

Examples of list of goals are described above. Examples of how progress towards meeting the goals is measured are described herein, according to some implementations. When a goal includes a specific duration/timeframe for meeting that goal, some implementations have a general set of default values that may be used to measure progress on meeting the goal (for instance, 15% progress towards achieving the goal from the baseline value when 25% of the duration/timeframe has passed, 40% progress towards achieving the goal from the baseline value when 50% of the duration/timeframe has passed, 70% progress towards achieving the goal from the baseline value when 75% of the duration/timeframe has passed, and 100% progress towards achieving the goal from the baseline value when 100% of the duration/timeframe has passed). In some implementations, the default values set may be customized or modified to meet each individual's needs for tracking progress to each goal.

Examples for providing feedback to motivate a person are described herein, according to some implementations. Some implementations train, advise, recommend and encourage users to set goals using an implementation (sometimes called the SMART framework) for goal setting originally described by George Doran in the November 1981 issue of Management Review-namely each goal should be Specific, Measurable, Achievable, Relevant, and Time-bound. The use of SMART goals reduces misunderstandings and confusions by all involved regarding what goal is trying to be achieved, how it is to be completed, and when it is expected to be completed by, providing the user trying to achieve the goal motivation to achieve the very specific targets. In addition, since the user will be able to share his/her goals with personal support team and clinical care team members, the user may be more motivated through feedback such as bio feedback or to see correlations to information they did not understand before but are now reflected. An example is a relationship to a late meal or having alcohol before bed and their impact on quality of sleep, resting heart rate, etc. The patients may be motivated to adopt better daily life practices and to not fail in achieving those goals since others will be able to track the user's progress, and the personal and clinical care team members can provide further motivation to the user to achieve the goals.

In some implementations, the method further includes: integrating and orchestrating services and analytics to enable collaborative, integrated planning for health, wellness, fitness, and athletic-performance, change monitoring and measurement, behavior incenting, coaching, supporting, and reporting both internal to the platform and to external authorized recipients. In some implementations, data-retrieval service retrieves medical data from FHIR-based APIs, physician portals, uploaded content, and digital personal health-monitoring devices. Data retrieved from FHIR-based APIs are directly integrated into SPHERE℠. Data retrieved from physician portals may be extracted, transformed into FHIR resources, and loaded (ETL) into a person's SPHERE℠ record. Using a collaboration channel, the person and their DREAMS™ Team may upload content and digital personal health-monitoring device data, which are then transformed into FHIR resources and then loaded (ETL) into the person's SPHERE℠ record. A person, along with their DREAMS™ team may use the collaboration channel and DREAMS™ tools to retrieve care plans from SPHERE℠ and use them to create the person's DREAMS™ Plan, where it is persisted as a FHIR resource in SPHERE℠. The collaboration channel and DREAMS™ engine may also be used to create person-specific surveys. A person who logs into the platform may be presented with DREAMS™ options to review their DREAMS™ Plan, create or update a schedule, respond to reminders, or complete a survey. Survey data may be captured as FHIR resources and used to capture DREAMS™ plan data (e.g., metrics, diet update), SDOH data, preference data, or other uses as determined by the person, DREAMS™ team, providers, or other Vital eCare services (e.g., rules engine, machine learning, deidentified for population-based AI).

Some implementations provide a set of tools to set up conditions and rules for a variety of health conditions and vitals that are important to monitor. Using these conditions and rules for an individual patient or a population of patients, some implementations may monitor status for the conditions or guidelines. For example, if blood pressure spikes and weight increases unexpectedly and diet and exercise are omitted for the past week, the system may alert a predetermined set of authorized users as well as the patient.

In some implementations, the health and wellness plan includes one or more goals and associated timelines.

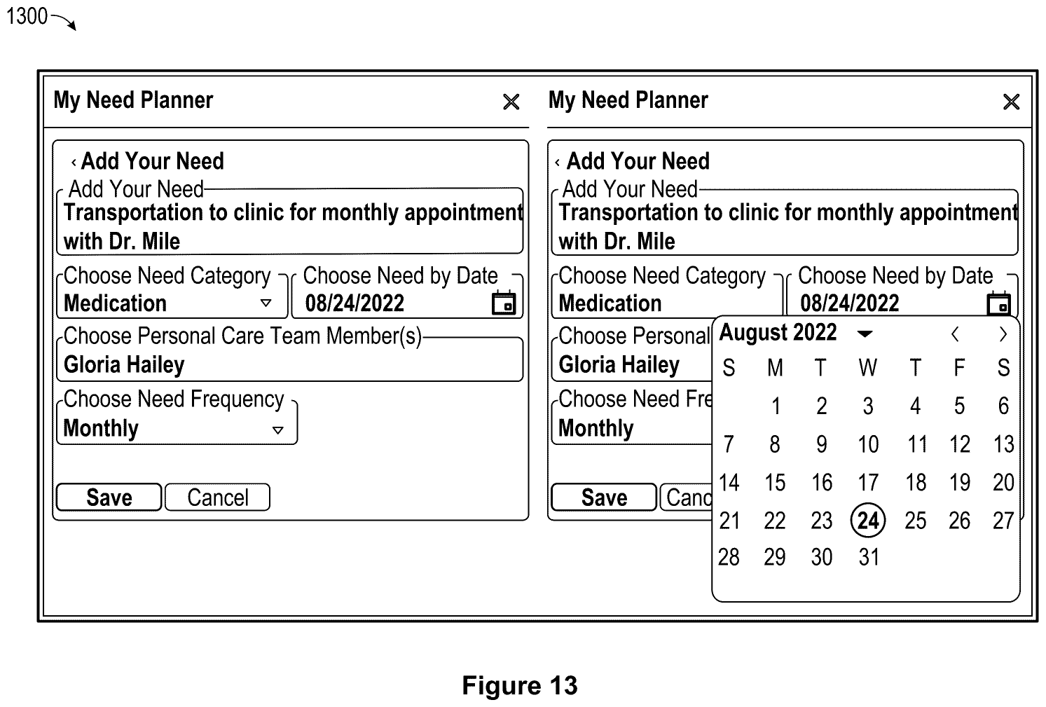

In some implementations, assigning the roles, responsibilities and role-based access rights includes: scheduling tasks, events and activities with calendar-based planning and management tools; managing task dependencies and task interdependencies; assigning of task, event and activity owners; and tracking of progress and completion of tasks, events and activities to support notification of appropriate users. FIG. 13 shows an example user interface 1300 of a calendar-based planning tool for needs activities, according to some implementations. Some tasks may be dependent on other tasks. For example, a task to perform blood glucose measurements prior to every meal is dependent on, and cannot be performed until the user obtains the necessary blood glucose monitoring equipment and supplies. Similarly, a task to adhere to a 2000 calorie ADA diet is dependent on the user or some other member of his/her support team being educated on and understanding the requirements of the diet and developing a compliant diet plan that the user would be comfortable implementing. Task interdependencies occur when different tasks impact each other. An interdependency example is that some medicines need to be taken with food, while other medicines need to be taken on an empty stomach, meaning that a user's medicine administration schedule needs to be designed keeping in mind that some medicines need to be taken within a certain time period before or after a meal, while other medicine need to be taken outside of a certain time period before or after a meal. The efficacy of the medication is critical in some cases to these conditions being met. With regards to calendar-based planning and management, a task represents a defined set of work with one or more specific goals that usually needs to be performed within a given timeframe, an activity represents an action that may not have a specific goal or timeframe within which to perform it by, and an event represents a specific instance of an activity. Examples of tasks include performing blood glucose measurements prior to every meal, weighing oneself on a daily basis, and filling out an attitude/lifestyle survey every two weeks survey. Examples of activities include taking one's medications as prescribed, wearing a device that tracks and monitors sleep quality and habits, and getting more rest. Examples of events include a clinic appointment with a specialist, a support group meeting, and a specific instance of a work-out/exercise session at a gym.

In some implementations, the method further includes: applying a rules-based decision algorithm, based on the health and wellness plans, the person's health system records and real-time health and wellness information, to trigger one or more updates selected from the group consisting of:
i. generating system alerts and providing notifications regarding missing information;
ii. identifying changes to the health and wellness plans, the person's health system records, and the real-time health and wellness information;
iii. notifying appropriate users when tasks, events and activities are completed, on pace with or falling behind expected progress, or that need to be completed;
iv. alerting the person and other authorized users of changes in team composition and/or role/access capabilities; and
V. suggesting new activities or changes to activities based on the rules-based engine and updated data.

Some implementations may use the following format for rules: If (expression) then (additional rule or activity) else (additional rule or activity). An expression is a statement that the rules engine can evaluate as either true or false (not true). If the expression is evaluated as true, the rules engine then goes to the “then” portion of the rule and evaluates the additional rule in the “then” portion or performs the “activity” indicated in the “then” portion. If the expression is evaluated as false, the rules engine then goes to the “else” portion of the rule and evaluates the additional rule in the “else” portion or performs the “activity” indicated in the “else” portion. The else portion of a rule is optional, meaning that rules can range from very simple rules to very complex, nested rules. Rules using this format are then built in the system using the system's rule building tools which allow the rule builder to create rules that follow this format. Examples of rules are shown below, according to some implementations:
If (user has not reported blood pressure readings for 3 days) then (send user a message reminding them to upload or enter their blood pressure readings or to check that their previously internet connected device connection to the platform needs to be reset).
If (task assigned to user is not completed and due in the next 3 days) then (send user and their proxy a message that a task that is due soon remains uncompleted).
If (user is designated as a Transitional Care Management patient and has been discharged in the past week and a face-to-face visit has occurred within 7 days of discharge) then (validate that a 99496 billing code exists for that user for that Transitional Care Management billing period) else (If (user is designated as a Transitional Care Management patient and has been discharged in the past 2 weeks and a face-to-face visit has occurred within 14 days of discharge) then (validate that a 99495 billing code exists for that user for that Transitional Care Management billing period)).

Some implementations may use decision-based algorithms created by nationally recognized societies and organizations, such as the American Heart Association Blood Pressure Treatment Algorithm and/or the American Diabetes Association Low Blood Sugar (Hypoglycemia) Treatment Algorithm. Some implementations may use decision-based algorithms developed or utilized by client organizations, such as a client specific algorithm to manage dementia, and/or a client developed or third party algorithm used by a client to manage patient weight loss.

In some implementations, the method further includes performing receipt, recognition, transformation, metadata tagging, and persistence of any structured or unstructured digital content. Some implementations use security technology to assure that any data coming in are safe. For example, some implementations check if the data contain no embedded executable code, including executable SQL. Safe data coming in are then processed through extract/transform/load (ETL) technology to safely extract relevant information, transform the data into structured (FHIR) resources that may then be consumed and/or loaded into SPHERE℠. Persons, and their Personal team members may be notified that new data are available. Surveys are structured documents that present questions in human-understandable format. Responses to survey questions may be captured directly into a format (FHIR) that is consumable by SPHERE℠. In some implementations, the system back-end is partitioned into several trust zones, and operated under a zero-trust model wherein exchanges between zones are structured and examined to assure that they are both safe and presented using appropriate, consumable formats.

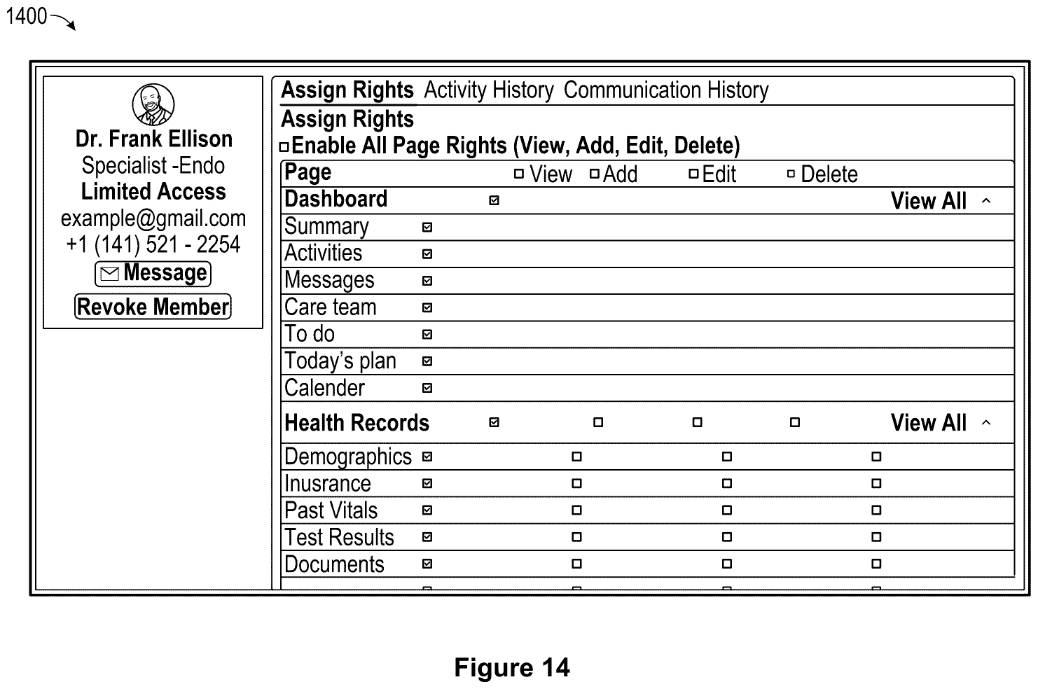

In some implementations, the method further includes: providing an interface to assign the roles, responsibilities and role-based access rights to data and services to individuals of the plurality of individuals; and receiving, via the interface, the roles, responsibilities and role-based access rights to data and services. Examples of interfaces are described above in reference to FIG. 9, according to some implementations. FIG. 14 shows another example user interface 1400, according to some implementations.

In some implementations, the method further includes providing a summary snapshot of current state and progress for each individual of the plurality of individuals and the Person, in accordance with the role-based access rights to data and services assigned to each individual.

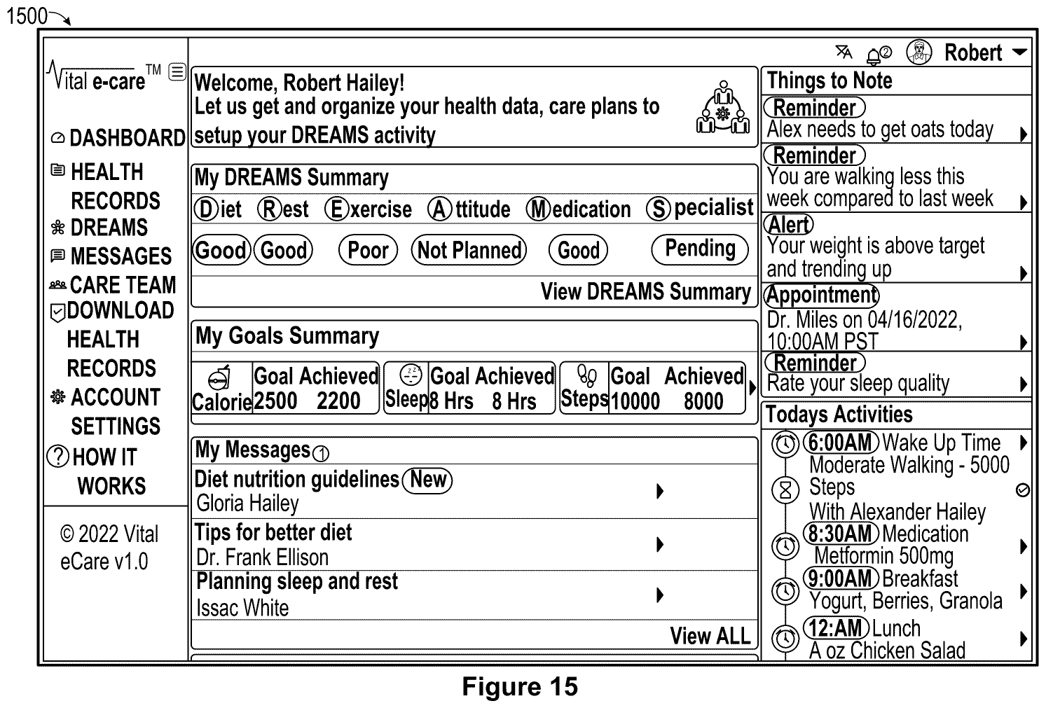

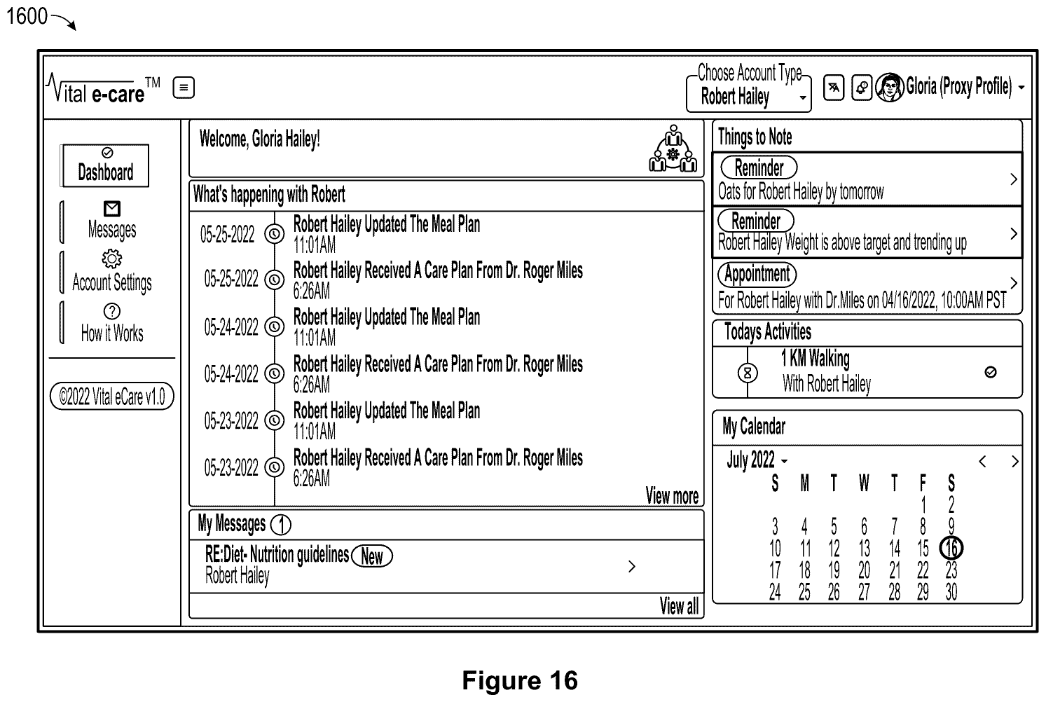

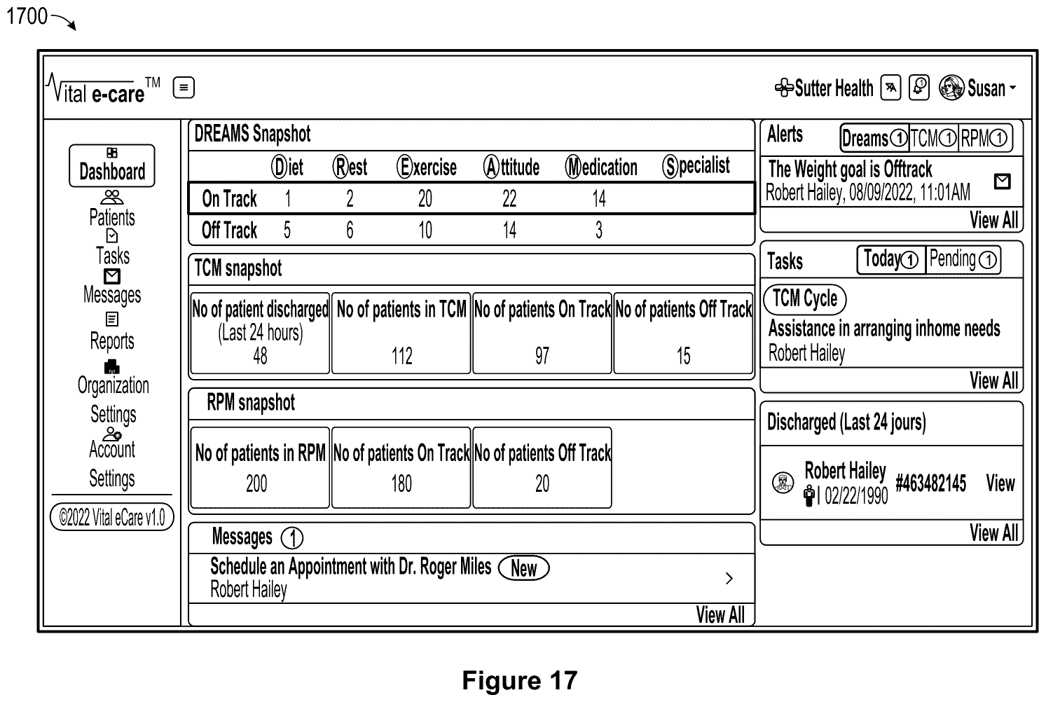

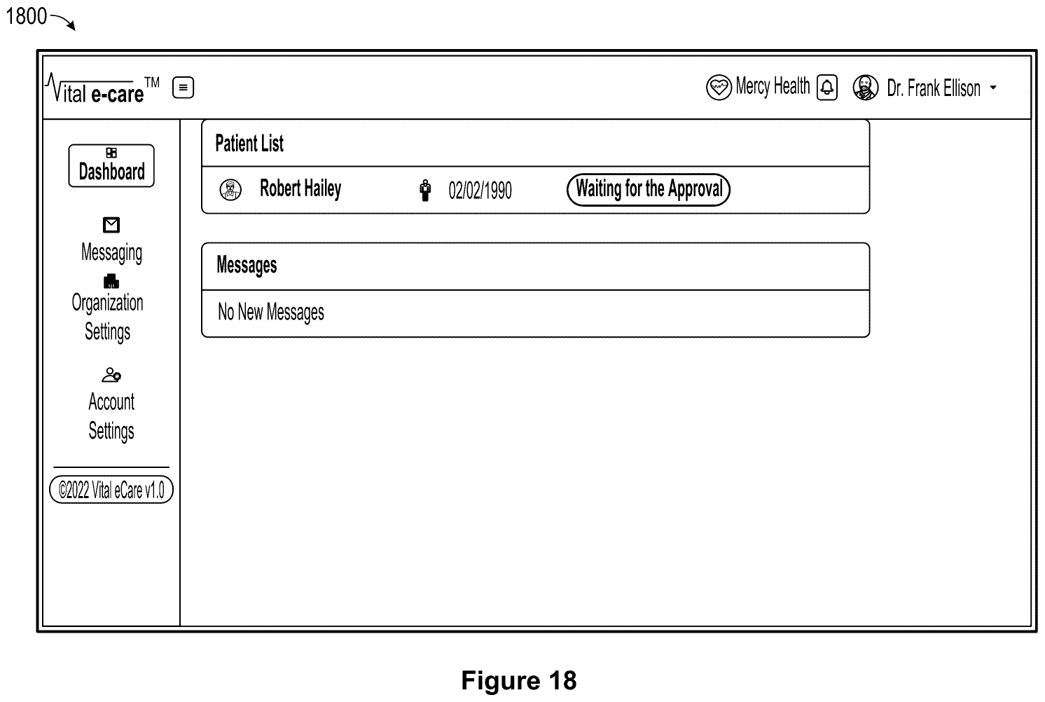

In some implementations, the summary snapshot is categorized based on whether an individual belongs to the Person's family and friends, a healthcare team, or a specialist team. FIGS. 15-18 show example visualizations of summaries (visualizations 1500, 1600, 1700, 1800, respectively), for different roles, according to some implementations.

In some implementations, the method further includes applying information security measures for protecting confidentiality, integrity and availability of data and services available to the Person and the plurality of individuals. Information security measures may be provided for protecting confidentiality, integrity and availability of data and services available to the Person and the plurality of individuals. Security protection builds up from a strong foundation. In some implementations, the platform is built on the concept of zero-trust. The user login portal is outside a firewall protecting all system data and services. User authentication and authorization services behind a first firewall may generate user tokens authorizing entry into specific trust zones. In some implementations, backend internal trust zones are behind a second firewall, each trust zone includes its own trusted network, and interactions may be allowed only over trusted (TLS) links. For examples, trust zones might separately protect managed microservices, data, and analytics. User entry into services provided by these trust zones is only through authorization specifically to those services. Data are integrity checked and encrypted for storage and are decrypted only for authorized use and re-integrity checked and encrypted for storage. Encryption keys are separately protected in an encrypted key vault. Application performance management tools are used to assure that microservices are performing as specified, with pipeline management technology supporting predictable, managed performance. In some implementations, strong role-based controls assure that administrative roles are separated and assigned only to individuals that are both qualified to perform the assigned functions and with proven trustworthiness. As described earlier, some implementations offer users the ability to assign pre-defined roles, or to define their own roles using individual authorizations. Actions of both privileged administrative users and application users may be recorded in audit trails and continuously reviewed using computer-aided audit techniques. Cloud security management tools may be used to continually monitor system operations to detect and address known system vulnerabilities, and to advise administrators of required changes from system events or new regulatory actions.

In some implementations, the method further includes: for each person of a plurality of persons: generating a respective health and wellness plan based on a respective repository of health and wellness data; selecting a respective plurality of individuals to form a respective support network for the respective person; assigning respective roles, responsibilities and role-based access rights for the respective plurality of individuals in the support network, based on the health and wellness plan; and reporting and updating (i) the respective health and wellness plan for the person, and (ii) the respective roles, responsibilities and role-based access rights for the plurality of individuals, based on tracking health of the respective person. Some implementations managing these aspects for different persons simultaneously, and/or asynchronously, for generating a respective health and wellness plan based on a respective repository of health and wellness data. Some implementations provide to the person's collaborators (sometimes called the DREAMS™ team) tools for retrieving from SPHERE℠ the individual provider care plans that have been prescribed for the person, and for prioritizing the individual assignments of these care plans and organizing them into a set of prioritized goals, with milestones and metrics for achieving those goals for each of the DREAMS™ domains (e.g., diet, rest, exercise, attitude/lifestyle/mental health, medical, and specialist) as part of the DREAMS™ Plan. Through a collaboration channel, the Personal team may discuss the DREAMS™ Plan with the Person who may agree, disagree, or modify the DREAMS™ Plan assignments. The collaboration channel offers both synchronous and asynchronous tools for accomplishing these actions. One challenge might be difficulties in getting the Personal team to take the required actions. One approach that may be useful in this event is the notification system, which may be used by a provider, specialist, person, or a Personal team member when such roadblocks occur. Notifications may be sent through the collaboration channel's messaging service, through the person's calendar, or back through the provider (or a provider system) associated with the person.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

What is claimed is:

1. A method for health and wellness management, comprising:
generating a health and wellness plan for a person that includes (i) a diet plan, (ii) a rest plan, (iii) an exercise plan, (iv) an attitude/lifestyle/mental health plan, (v) a medical care plan, and (vi) specialist services, based on a repository of health and wellness data that is specific to the person;
selecting a plurality of individuals to form a support network for the person based on the health and wellness plan;
assigning roles, responsibilities and role-based access rights for the plurality of individuals in the support network, based on the health and wellness plan; and
reporting and updating (i) the health and wellness plan for the person, and (ii) the roles, responsibilities and role-based access rights for the plurality of individuals, based on tracking health of the person.

2. The method of claim 1, further comprising:
generating the repository of health and wellness data for the person by integrating the person's health-system records with other digital content related to the person's health, wellness, conditioning, fitness, athletic performance, or professional physical, mental or other performance gathered from external sources, including care providers, athletic trainers, coaches, labs, specialists, and other individuals and organizations, along with genomic data, social determinants of health (SDOH), device data, financial, cost and/or claims data, and other person-generated data provided through uploads, responses to surveys generated, and outcomes feedback obtained during execution of the health and wellness plan

3. The method of any of claims 1-2, further comprising:
providing an always-on, private collaboration channel that enables sharing and exchange of content, for integrating and managing the health and wellness plan among the person and the plurality of individuals in the support network.

4. The method of any of claims 1-3, further comprising:
using an artificial intelligence (AI)-based knowledge engine to: (i) continuously receive data for the person, analyze a current state, identify data omissions and potential adverse events, and (ii) generate insights for modifying and/or fulfilling the health and wellness plan based on the current state and identified data omissions and potential adverse events; and
providing feedback to the person and the plurality of individuals, based on the generated insights.

5. The method of any of claims 1-4, further comprising:
analyzing, using an AI-based knowledge engine, anonymized data across a population including the person, to obtain population-level knowledge; and
providing feedback to the person and the plurality of individuals, based on the population-level knowledge.

6. The method of any of claims 1-5, further comprising:
measuring progress towards meeting goals of the health and wellness plan; and
providing feedback to motivate the person and modify daily behavior and activities of the person, to meet the goals, based on the measured progress.

7. The method of any of claims 1-6, further comprising:
integrating and orchestrating services and analytics to enable collaborative, integrated planning for health, wellness, fitness, and athletic-performance, change monitoring and measurement, behavior incenting, coaching, supporting, and reporting both internally and to external authorized recipients.

8. The method of any of claims 1-7, wherein the health and wellness plan includes one or more goals and associated timelines.

9. The method of any of claims 1-8, wherein assigning the roles, responsibilities and role-based access rights comprises:
scheduling tasks, events and activities with calendar-based planning and management tools;
managing task dependencies and task interdependencies;
assigning of task, event and activity owners; and
tracking of progress and completion of tasks, events and activities to support notification of appropriate users.

10. The method of any of claims 1-9, further comprising:
applying a rules-based decision algorithm, based on the health and wellness plans, the person's health system records and real-time health and wellness information, to trigger one or more updates selected from the group consisting of:
i. generating system alerts and providing notifications regarding missing information;
ii. identifying changes to the health and wellness plans, the person's health system records, and the real-time health and wellness information;
iii. notifying appropriate users when tasks, events and activities are completed, on pace with or falling behind expected progress, or that need to be completed;
iv. alerting the person and other authorized users of changes in team composition and/or role/access capabilities; and
v. suggesting new activities or changes to activities based on the rules-based decision algorithm and updated data.

11. The method of any of claims 1-10, further comprising:
performing receipt, recognition, transformation, metadata tagging, and persistence of any structured or unstructured digital content.

12. The method of any of claims 1-11, further comprising:
providing an interface to assign the roles, responsibilities and role-based access rights to data and services to individuals of the plurality of individuals; and
receiving, via the interface, the roles, responsibilities and role-based access rights to data and services.

13. The method of any of claims 1-12, further comprising:
providing a summary snapshot of current state and progress for each individual of the plurality of individuals and the person, in accordance with the role-based access rights to data and services assigned to each individual.

14. The method of claim 13, wherein the summary snapshot is categorized based on whether an individual belongs to the person's family and friends, a healthcare team, or a specialist team.

15. The method of any of claims 1-14, further comprising:
applying information security measures for protecting confidentiality, integrity and availability of data and services available to the person and the plurality of individuals.

16. The method of any of claims 1-15, further comprising:
for each person of a plurality of persons:
generating a respective health and wellness plan based on a respective repository of health and wellness data;
selecting a respective plurality of individuals to form a respective support network for the respective person;
assigning respective roles, responsibilities and role-based access rights for the respective plurality of individuals in the support network, based on the health and wellness plan; and
reporting and updating (i) the respective health and wellness plan for the person, and (ii) the respective roles, responsibilities and role-based access rights for the plurality of individuals, based on tracking health of the respective person.

17. A computing device, comprising:
one or more processors;
memory;
a display; and
one or more programs stored in the memory and configured for execution by the one or more processors, the one or more programs comprising instructions for:
generating a health and wellness plan for a person that includes (i) a diet plan, (ii) a rest plan, (iii) an exercise plan, (iv) a mental health plan, (v) a medical care plan, and (vi) specialist services, based on a repository of health and wellness data that is specific to the person;
selecting a plurality of individuals to form a support network for the person based on the health and wellness plan;
assigning roles, responsibilities and role-based access rights for the plurality of individuals in the support network, based on the health and wellness plan; and
reporting and updating (i) the health and wellness plan for the person, and (ii) the roles, responsibilities and role-based access rights for the plurality of individuals, based on tracking health of the person.

18. The computing device of claim 17, wherein the one or more programs further comprise instructions for:
providing an always-on, private collaboration channel that enables sharing and exchange of content, for integrating and managing the health and wellness plan among the person and the plurality of individuals in the support network.

19. The computing device of any of claims 17-18, wherein the one or more programs further comprise instructions for:
using an artificial intelligence (AI)-based knowledge engine to: (i) continuously receive data for the person, analyze a current state, identify data omissions and potential adverse events, and (ii) generate insights for modifying and/or fulfilling the health and wellness plan based on the current state and identified data omissions and potential adverse events; and
providing feedback to the person and the plurality of individuals, based on the generated insights.

20. A non-transitory computer-readable storage medium storing one or more programs configured for execution by a computing device having one or more processors, memory, and a display, the one or more programs comprising instructions for:
generating a health and wellness plan for a person that includes (i) a diet plan, (ii) a rest plan, (iii) an exercise plan, (iv) a mental health plan, (v) a medical care plan, and (vi) specialist services, based on a repository of health and wellness data that is specific to the person;
selecting a plurality of individuals to form a support network for the person based on the health and wellness plan;
assigning roles, responsibilities and role-based access rights for the plurality of individuals in the support network, based on the health and wellness plan; and
reporting and updating (i) the health and wellness plan for the person, and (ii) the roles, responsibilities and role-based access rights for the plurality of individuals, based on tracking health of the person.
